(19) United States
(12) Patent Application Publication
(10) Pub. No.: US 2024/0091285 A1
(43) Pub. Date: Mar. 21, 2024

(54) COMPOSITIONS AND METHODS OF TREATMENT OF TUMORS EXPRESSING PUTATIVE ZIKA VIRUS RECEPTOR PROTEINS

(71) Applicant: THE NEMOURS FOUNDATION, Jacksonville, FL (US)

(72) Inventors: Kenneth Andrew ALEXANDER, Orlando, FL (US); Rajarajeshwari VENKATARAMAN, Orlando, FL (US); Peter Eugene PHELAN, III, Palm Bay, FL (US)

(73) Assignee: THE NEMOURS FOUNDATION, Jacksonville, FL (US)

(21) Appl. No.: 18/368,737

(22) Filed: Sep. 15, 2023

Related U.S. Application Data
(60) Provisional application No. 63/536,790, filed on Sep. 6, 2023.
(60) Provisional application No. 63/407,480, filed on Sep. 16, 2022.

(51) Int. Cl. A61K 35/768; A61K 9/127; A61K 45/06; A61P 35/00

(52) U.S. Cl. CPC A61K 35/768; A61K 9/127; A61K 45/06; A61P 35/00

(57) ABSTRACT

A pharmaceutical composition for treatment of a tumor expressing at least one of a plurality of receptor proteins including CD24, Axl, DC-SIGN, Tyro3, MER, MerTK TIM-1, TIM-4, TLR3, TLR8, RIG-1, and MDA5. The pharmaceutical composition includes either a purified viral RNA of an oncolytic Zika virus in a liposome and a pharmaceutically acceptable carrier, or a pharmaceutically acceptable carrier and a unit dosage form of an oncolytic Zika virus.

### CROSS REFERENCE TO RELATED APPLICATIONS

This application claims the benefit of the filing date of two previously filed provisional applications: U.S. Provisional Application No. 63/536,790, filed on Sep. 6, 2023, and U.S. Provisional Application No. 63/407,480, filed on Sep. 16, 2022, which are both hereby incorporated by reference herein in their entireties.

### REFERENCE TO A SEQUENCE LISTING

The RNA used to transfect SKOV3 cancer cells was purified from isolated and purified Zika virus, strain MR766. The sequence is found at GenBank MW143022.1.

### FIELD

The disclosure relates to the field of treatment of cancer, and to diseases associated with tumors expressing a variety of putative Zika virus receptor proteins.

### BACKGROUND

Children and adults develop tumors that arise from various types of cells. Among various other types of cancers, ovarian cancer may be developed in women. Ovarian cancer originates from ovarian epithelial cells, the germ cells, and the gonadal stroma. Various types of ovarian cancers are derived from each cell type. For example, serous epithelial ovarian cancer, endometroid epithelial ovarian cancer, clear cell epithelial ovarian cancer, mucinous epithelial ovarian cancer, mucinous epithelial cancer, and undifferentiated epithelial ovarian cancers are all derived from epithelial cells cancers. Epithelial ovarian cancer is the most commonly occurring ovarian cancer as it accounts for approximately 90% of ovarian cancers. Approximately 75% of epithelial ovarian cancers are high-grade serous ovarian carcinomas. These are characterized by cancer cells that grow and spread faster than low-grade cancer cells. In these instances, if the cancer is detected at stage 3 or stage 4, the cancer is defined as a metastatic cancer. The term metastatic refers to cancers which have spread from the location at which the cancer started to a secondary or distant location within the body.

Ovarian germ cell tumors derive from the germ cells of the ovaries. There are various types of the ovarian germ cell tumors that may develop, including dysgerminoma which is the most commonly occurring ovarian germ cell tumor. Further, ovarian germ cell cancers may also include immature ovarian teratoma and endodermal sinus tumors. Gonadal stromal ovarian tumors may be derived from gonadal cells within the ovaries. Gonadal stromal ovarian tumors are generally uncommon neoplasms and account for approximately 5% of ovarian tumor malignancies in women between the ages of 15 years old and 24 years old.

Ovarian cancer accounts for approximately 2.5% of cancers in women and is the fifth leading cause of cancer-related death among women. Ovarian cancer is typically treated through combinations of surgery and chemotherapy. Surgical treatments can require oophorectomy, omentectomy, salpingectomy, hysterectomy, and pelvic and retroperitoneal lymph node dissection/resection. Chemotherapy may be used in conjunction with surgery and/or without the surgery. While various different types of chemotherapy drugs may be used for treating ovarian cancer, some of the more prevalent types of chemotherapy drugs for treating ovarian cancer may include doxorubicin and liposomal doxorubicin, topotecan, gemcitabine, niraparib and other PARP inhibitors, bevacizumab, paclitaxel and other taxanes, bleomycin, etoposide, cisplatin, and carboplatin. While some types of ovarian cancers may be sensitive to these chemotherapies, some types of ovarian cancers may be resistant to chemotherapy. In some cases, continuous treatment of the cancer with chemotherapy causes the cancer cells to become resistant to the chemotherapy. In other words, the administration of chemotherapies to the tumor may not treat the tumor successfully. Additionally, in the instances where chemotherapy may be effective, there are many side effects that may accompany chemotherapy, including but not limited to, immunosuppression, infection, fatigue, nausea and vomiting, mucositis, alopecia, and poor appetite.

Radiation therapy is another method of treatment that may be used for treating ovarian cancer. However, radiation therapy may also be accompanied with various side effects, including but not limited to skin changes, fatigue, nausea, and vomiting. Further, hormone therapy is also used for treating ovarian cancer. This treatment is often used for treating stomal ovarian cancers however, it is rarely used for treating epithelial ovarian cancers which are the most commonly occurring ovarian cancers.

What is needed is an alternative, effective treatment for diseases characterized by tumors, such as ovarian cancer tumors, that address the shortcomings of the existing treatments.

### SUMMARY

As envisioned in the present application with respect to the disclosed compositions of matter and methods, in one aspect the embodiments of the disclosure comprise the components and/or steps disclosed herein. In another aspect, the embodiments of the disclosure consist essentially of the components and/or steps disclosed herein. In yet another aspect, the embodiments of the disclosure consist of the components and/or steps disclosed herein.

Furthermore, it is to be understood that the description that follows encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group.

### DESCRIPTION OF THE FIGURES

The patent or application file contains at least one drawing executed in color. Copies of this patent or patent application publication with color drawing(s) will be provided by the Office upon request and payment of the necessary fee.

FIG. 1 depicts cell viability of human ovarian cancer cells infected by Zika virus over a four day period. All infections were performed at multiplicity of infection (MOI)=1 and the results shown are normalized to uninfected control cells for each cell line.

FIG. 2 depicts images of Zika virus infected human ovarian cell lines seventy-hours after infection compared to the human ovarian cell lines without infection by Zika virus.

FIG. 3 depicts Western blot analysis for expression of NS1 and envelope proteins of various human ovarian cell lines after infection of the cell lines with Zika virus.

FIG. 4 depicts cell viability of A2780 human ovarian cells having resistance to cis-platinum (A2780-cis) and to doxorubicin (a.k.a. Adriamycin)(A2780-ADR) and having sensitivity to cis-platinum and to doxorubicin over a four-day period. All infections were performed at multiplicity of infection (MOI)=1 and the results shown are normalized to noninfected cell lines.

FIGS. 5 and 6 depict CD24 and AXL mRNA expression levels in (cis-platinum-sensitive) A2780, isogenic (cis-platinum-resistant) A2780-cis, isogenic (doxorubicin-resistant) A2780-ADR ovarian cancer cells, and cis-platinum- and doxorubicin-sensitive ES2 cells.

FIG. 7 depicts the tumor size of SkOV3 ovarian cancer tumors in Nod-SCID-interferon gamma-deficient (NSG) immunodeficient laboratory mice after being injected with Zika virus or a control vehicle injection. The tumor size is monitored over 38 days, with Zika virus and vehicle injections completed on Day 15.

FIG. 8 depicts the tumor weight of SkOV3 ovarian cancer tumors in NSG immunodeficient laboratory mice after being injected with Zika virus or with a vehicle control injection. The weight measurement is taken at Day 38. The injections were administered at Day 15.

FIG. 9 depicts the tumor growth fold change of in vivo SkOV3 ovarian cancer tumors after injection with Zika virus or injection with a vehicle solution over the course of 17 Days.

FIG. 10 depicts representative immunohistochemistry staining at 200× of isolate SkOV3 tumors stained for Zika virus NS2B protein after infection with Zika virus or without infection with Zika virus.

FIGS. 11A and 11D depict images taken at 4× magnification and 20× magnification, respectively, of SkOV3 cells on Day 4 after transfection with 0.1 ug of RNA encoding green fluorescent protein (GFP).

FIGS. 11B and 11E depict images taken at 4× magnification and 20× magnification, respectively, of SkOV3 cells on Day 4 after injection with Zika virus strain MR766 at multiplicity of infection (MOI)=3.

FIGS. 11C and 11F depict images taken at 4× magnification and 20× magnification, respectively, of SkOV3 cells on Day 4 after transfection with purified Zika virus strain MR766 RNA at 0.83 RNA genome copies per cell.

FIGS. 12A and 12D depict images taken at 4× magnification and 20× magnification, respectively, of SkOV3 cells on Day 6 following transfection with 0.1 ug of RNA encoding GFP.

FIGS. 12B and 12E depict images taken at 4× magnification and 20× magnification, respectively, of SkOV3 cells on Day 6 following infection with Zika virus strain MR766 at multiplicity of infection (MOI)=3.

FIGS. 12C and 12F depict images taken at 4× magnification and 20× magnification, respectively, of SkOV3 cells on Day 6 following transfection of purified Zika strain MR766 RNA at 0.83 genome copies per cell.

FIG. 13A depicts tumor volumes as a function of virus treatment for Zika virus strains MR766, Brazil-SJRP/2016-184, and Nicaragua/2016 UCB 7420 that were injected into subcutaneous SKOV3 tumors created in female NSG mice.

FIG. 13B depicts tumor volumes as a function of virus treatment for Zika virus strains MR766, Brazil-SJRP/2016-184, and Nicaragua/2016 UCB 7420 that were injected into subcutaneous OVCAR8 tumors created in female NSG mice.

FIG. 13C depicts H&E and viral NS2B staining (red) of virus-treated OVCAR8 tumors.

FIG. 14A depicts cell death reported as fluorescence units for rhabdomyosarcoma (RD) cells infected with MR766 viruses.

FIG. 14B depicts 20× brightfield microscopy images of Zika virus MR766-infected RD cells.

FIG. 14C depicts 20× brightfield microscopy images of Zika virus Nicaragua-infected RD cells.

FIG. 15 depicts western blots for RD cells infected with Zika virus MR766.

FIG. 16 depicts results from plaque assays of tissue culture supernatants of RD cells that were infected with Zika virus MR766.

FIG. 17 depicts the results of a RT-PCT comparison between AXL and CD24 mRNA expression with mRNA expression of the housekeeping gene RPS18.

### DEFINITIONS

Unless defined otherwise, all technical and scientific terms used herein have the same meaning as commonly understood by one of ordinary skill in the art to which the disclosure pertains.

Although any methods and materials similar or equivalent to those described herein can be used in the practice for testing of the present disclosure, the preferred materials and methods are described herein. In describing and claiming the present disclosure, the following terminology will be used. It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

As used herein, the term “and/or” includes any and all combinations of one or more of the associated listed items.

“About” as used herein when referring to a measurable value such as an amount, a temporal duration, and the like, indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters. As used herein, the term “about” means that the number being described can deviate by plus or minus five percent of the number. For example, “about 250 g” means from 237.5-262.5 g. When the term “about” is used in a range, then the lower limit may be as much as minus 5% of the lower number and the upper limit may extend up to plus 5% of the upper number. For example, a range of about 100 to about 200 g indicates a range that extends from as low as 95 g up to 210 g.

An “effective amount” as used herein, means an amount which provides the intended effect. For an oncolytic virus used to treat or ameliorate a tumor, an effective amount is an amount of the virus sufficient to alleviate or eliminate the symptoms of the tumor or to slow down the progression of the tumor in a subject. It is understood, however, that the full therapeutic effect does not necessarily occur by administration of one dose and may occur only after administration of a series of doses. Thus, an effective amount may be administered in one or more administrations. In the context of therapeutic or prophylactic applications, the amount of active agent administered to the subject will depend on the type and severity of the disease or condition and on the characteristics of the subject, such as general health, age, sex, body weight and tolerance to drugs. It will also depend on the degree, severity and type of disease or condition. The skilled artisan will be able to determine appropriate dosages depending on these and other factors. For example, in some instances, for subcutaneous infection of Zika virus an effective dose may range between approximately 100 and approximately 100,000,000 plaque-forming units of virus. Doses on the lower end of the range (i.e., approximately 100 plaque-forming units) may induce infection with consequent viremia but does on the high-end of the range (i.e., approximately 100,000,000 plaque-forming units may have more rapid oncolytic effects.

As used herein, “individual” or “patient” or “subject” (as in the subject of the treatment) means both mammals and non-mammals. Mammals include, for example, humans; non-human primates, e.g., apes and monkeys; cattle; horses; sheep; and goats. Non-mammals include, for example, fish and birds.

As used herein, “level of amplification” with reference to a particular gene means the gene's copy number in the genome of a cell. A copy number of about 10-fold or more above the copy number of a gene in normal cells of a corresponding normal tissue means that the gene is “amplified”.

As used herein, the term “pharmaceutically acceptable” refers to a formulation of a compound that does not significantly abrogate the biological activity, a pharmacological activity and/or other properties of an active agent when the formulated compound is administered to a patient. In certain embodiments, a pharmaceutically acceptable formulation does not cause significant irritation to a patient. Further, “pharmaceutically acceptable” in relation to a virus or virus derivative would be approved good manufacturing process (GMP) by the Food and Drug Administration (FDA), or a similar governing body of another country.

To “treat” a disease as the term is used herein, means to reduce the frequency or severity of at least one sign or symptom of a disease or disorder experienced by a subject. Treating may include the postponement of further disease progression, or reduction in the severity of symptoms that have or are expected to develop, ameliorating existing symptoms and preventing additional symptoms. “Treat” a tumor means alleviating or eliminating the symptoms of a tumor, reducing the size or extent of a tumor, reducing the number or sizes of tumor metastases, or slowing down the progression of the tumor. The alleviation is preferably at least about 10%, more preferably at least about 20%, 30%, 40%, 50%, 60%, 70%, 80%, 90% or 95%.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

Embodiments of the materials and methods are described below.

It has been found that Zika virus effectively kills tumor cells expressing the marker, CD24 (also known as CD24A, signal transducer C24A; see e.g., GenBank FJ226006 for the nucleotide sequence and ACI46150.1 for the protein sequence and also GenBank NG_041768.1; see also L. Wang et al., “A dinucleotide deletion in CD24 confers protection against autoimmune diseases,” PLoS Genet. 3(4): E49, 2007) or expressing the marker AXL. “AXL is a member of the TAM family with the high-affinity ligand growth arrest-specific protein 6 (GAS6).” Zhu, C., Wei, Y. & Wei, X. AXL receptor tyrosine kinase as a promising anti-cancer approach: functions, molecular mechanisms, and clinical applications. Mol Cancer 18, 153 (2019). https://doi.org/10.1186/s12943-019-1090-3. Further, various other receptors apart from Axl and CD24 expressed by tumor cells may be targeted and killed by Zika virus. For example, Zika virus may kill tumor cells expressing DC-SIGN, Tyro3, TIM1, TIM4, TLR3, TLR8, RIG-1, MDA5 and the MER tyrosine kinase (MerTK) as these are additional receptors of Zika virus. While the disclosure herein refers largely to the treatment of CD24 or AXL positive tumors, the tumors treated may express any of the putative receptor proteins listed above.

Zika virus infections in children and adults are mild. Approximately 80% of Zika virus infections in children and adults are asymptomatic. Patients with symptomatic Zika virus infections typically experience mild and spontaneously resolving symptoms including rash, fever, and conjunctivitis. Severe symptoms from Zika virus disease are rare. Zika viruses are not known to cause chronic infection. Severe Zika virus disease in immunocompromised individuals is rare.

A Brazilian strain of Zika virus infection has been linked to major birth defects, including microcephaly in the babies of women who are pregnant at the time they are infected with the virus and has been tied to an increase to the number of cases of Guillain-Barre syndrome in adults. Fetal injury appears to be more likely when maternal infection occurs during the first trimester. Most late-pregnancy maternal Zika infections rarely result in an overtly injured infant.

Based on the clinical observation that Zika virus infections in children and adults are most often asymptomatic, wild-type or genetically modified or attenuated Zika viruses or purified RNA or RNA derivatives encoding Zika virus in part or in whole may be safely and effectively therapeutically administered to children or adults with tumors that express CD24 alone or AXL as well. The tumors may additionally express any of the other putative Zika virus receptors described herein, including, but not limited to, DC-SIGN, Tyro3, TIM1, TIM4, TLR3, TLR8, RIG-1, MDA5 and the Mer tyrosine kinase.

The infecting Zika viruses will cause tumor lysis with little or no effect on normal host cells. This specificity of tumor cell lysis provides a safe, effective, and novel means for treating Zika virus-sensitive malignancies. It is believed that Zika viruses can be used to eliminate or reduce tumors expressing CD24 with minimal long-term adverse effect. CD24 positive tumors include (but are not limited to) neuroblastomas, ovarian cancer, colorectal cancer, B cell lymphomas, erythroleukemia, gliomas, non-small cell lung cancer, esophageal squamous cell carcinoma, hepatocellular carcinoma, hepatoblastoma, cholangiocarcinoma, pancreatic adenocarcinoma, urothelial carcinoma, breast cancer, primary neuroendocrine carcinoma, some breast cancers, and prostate carcinomas. Z. Fang et al., “CD24: From A to Z,” Cell. & Mol. Immun. 7: 100-13 (2010).

In addition, it is believed that Zika viruses can be used to reduce tumors from pediatric rhabdomyosarcoma. Rhabdomyosarcoma (RD) is a type of cancer that forms in soft tissues, primarily muscles. Rhabdomyosarcoma is the most common soft tissue sarcoma in children. There are several subtypes of rhabdomyosarcoma, but the two main categories are embryonal rhabdomyosarcoma and alveolar rhabdomyosarcoma. Pediatric rhabdomyosarcomas carry a poor prognosis, particularly metastatic rhabdomyosarcomas and rhabdomyosarcomas involving the head and neck. Treatments for pediatric rhabdomyosarcomas have not changed in decades, and outcomes for children with rhabdomyosarcomas have not improved.

The range of subjects treated with a composition comprising an oncolytic Zika virus (wild type or attenuated) includes children and adults with CD24-positive tumors. Pregnant females having a CD24-positive cancer may also be treated with Zika virus, however women in the first trimester of pregnancy would be excluded because of the risks of fetal harm. Also contemplated are compositions and methods of treating a pregnant woman who is believed to have been exposed to Zika virus or who resides in an area where Zika is prevalent with a neutralizing antibody or composition that will prevent Zika virus from interacting with CD24 as well as other high-risk immunocompromised individuals.

Because many CD24-expressing tumors (e.g., non-small cell lung cancer, prostate cancer, breast cancer, ovarian carcinomas, gliomas, and glioblastomas) are resistant or refractory to currently available cancer treatments, and because Zika viruses cause lysis of CD24-expressing tumors, Zika virus oncolytic therapy would be effective for treatment of many chemotherapy-resistant tumors.

As is the case with any potential cancer therapy, the risks of Zika virus infection (which are minimal) must be balanced against both the toxicity and poor efficacy of current chemotherapy. It should be noted that in some instances, chemotherapy-resistant tumors often carry a worse prognosis; thus, a Zika virus therapy may be ideal for otherwise treatment-refractory tumors.

There is no known passive human-to-human transmission of Zika virus infection. Human-to-human Zika virus transmission occurs in three ways:
1) Transmission by mosquito, whereby a competent strain of Aedes mosquito acquires Zika virus from an infected host, and then transmits the virus to a susceptible host.
2) Sexual transmission of Zika virus has been described, wherein a Zika virus-infected individual can transmit Zika virus to a sexual partner
3) Zika virus transmission through infusion of Zika virus-contaminated blood products has been reported
   Because there is no passive transmission of Zika virus, Zika viruses used clinically would present negligible risks to healthcare providers. To prevent the release of Zika virus into the community, it is recognized that therapy would be performed in an environment free of competent Aedes mosquitos. Patients receiving Zika virus therapy would be isolated in an environment free of competent Aedes mosquitos until the patient's virus levels in blood are undetectable. Zika virus-treated patients would also avoid sexual contact until there is no risk of sexual transmission. Zika virus-treated individuals would be ineligible to donate blood until the in individual no longer has circulating Zika virus.

Following infection, an oncolytic Zika virus can kill a susceptible cancerous cell by direct lytic infection, induction of apoptosis, induction of autophagy, or by initiating an immune response to viral antigens. The effect of the oncolytic virus is thus not limited to a single input dose. Susceptible cells can undergo a multi-cycle infection, resulting in the production of large numbers of progeny virus. These progeny virions can spread either locally to adjacent tumor cells, or systemically (i.e., by hematogenous spread) to distant metastatic sites. This feature of oncolytic therapy is particularly attractive for the treatment of inaccessible tumors, widespread metastases, or un-diagnosed micro-metastases.

Because Zika viruses are known to cross the blood-brain barrier, and because many central nervous system tumors express CD24, as well as Axl and other putative Zika virus receptors, for example including DC-SIGN, Tyro3, TIM1, TIM4, TLR3, TLR8, RIG-1, MDA5 and the Mer tyrosine kinase, Zika viruses may be effective for treatment of central nervous system tumors.

Accordingly, a method for treating diseases in a subject is provided by administration of an oncolytic Zika virus, where cells caused by the disease are characterized by the expression of CD24. Further, a method for treating diseases in a subject is provided by administration of an oncolytic Zika virus, where cells caused by the disease are characterized by the expression of CD24 and by the expression of Axl, or by other putative Zika virus receptor proteins (e.g., DC-SIGN, Tyro3, TIM1, TIM4, TLR3, TLR8, RIG-1, MDA5 and the Mer tyrosine kinase). Further, a method for treating diseases in a subject is provided by administration of an oncolytic Zika virus, where cells caused by the disease are characterized by the expression of Axl or other putative Zika virus receptor proteins. Even further, a method for treating the cancer cells which have developed a resistance to chemotherapy, for example cis-platinumdiaminedichloride-(herein after termed “cis-platinum” or “cis-platin”), carboplatin, doxorubicin-, or epidermal growth factor inhibitor-resistant tumors, will be described herein. It has been determined that these chemotherapy-resistant tumor cells may have increase expression of CD24 and/or Axl (or other putative Zika virus receptor proteins, for example DC-SIGN, Tyro3, TIM1, TIM4, TLR3, TLR8, RIG-1, MDA5 and the Mer tyrosine kinase). While the disclosures herein may be made primarily with reference to ovarian cancer cells, the methods and compositions described herein may be used for treatment of any other type of cancer or disease associated with the expression of CD24 and/or Axl. The subject may be a mammal, particularly one selected from the group consisting of dogs, cats, sheep, goats, cattle, horses, pigs, humans, and non-human primates. The mammal is preferably human.

Tumors treatable comprise those that are populated by tumor cells which express CD24 or Axl or other putative Zika virus receptor proteins. Tumors that can be treated using the compositions and methods disclosed herein include tumors that are refractory to treatment with chemotherapeutics. The term “refractory”, when used herein in reference to a tumor, refers to a tumor (and/or metastases thereof) that shows no or only weak anti-proliferative response (i.e., or only a weak inhibition of tumor growth) after treatment with at least one chemotherapeutic agent. Thus, a refractory tumor cannot be treated at all or only with unsatisfying results with at least one (preferably standard) chemotherapeutic agent. Treatment of refractory tumors as mentioned is to be understood to encompass not only (1) tumors where one or more chemotherapeutics have already failed during treatment of a patient, but also (2) tumors that can be shown to be refractory by other means, e.g., biopsy and culture in the presence of chemotherapeutics and radiation treatment.

As discussed, and shown in the examples herein, the refractory tumors encompassed by those where one or more chemotherapeutics have already failed during treatment of a patient, may be more susceptible to treatment by Zika virus than those that are sensitive to the chemotherapeutics. Examples of the chemotherapeutics that the refractory tumors may be resistant to include, but are not limited to, cis-platinum and its derivatives, doxorubicin, mTOR inhibitors (e.g., rapamycin, rapalogues, torin1, dactolisib), agents that inhibit mTOR by activation of adenosine monophosphate-activated protein kinase (e.g., metformin, trehalose, resveratrol), and statins (e.g., simvastatin, atorvastatin). This resistance to the chemotherapeutics may be due to an increased expression of CD24 and/or Axl (or other putative Zika virus receptor proteins) from these tumor cells. It is proposed that the increase in CD24 expression and/or Axl expression (and/or expression of other putative Zika virus receptor proteins) may be due to the cells being driven into a state of autophagy after continued treatment with chemotherapeutics. Similarly, tumor cells that have been treated with radiotherapy may also exhibit an increased CD24 expression and/or Axl expression (and/or expression of other putative Zika virus receptor proteins) due to the tendency of radiation to induce autophagy of the treated cells. As such, tumors being treated with radiotherapy may also be of interest for treatment through lysis by Zika virus infection.

Further, it has been observed that cancer cells of tumors that have been developed a resistance to chemotherapeutics often take on a stem cell-like phenotype. This is often associated with the increased Axl and CD24 expression. The stem cell-like phenotype is also frequently associated with an increased tendency toward metastasis, faster cell and tumor growth rates, and more invasiveness within the body. In other words, the stem cell-like phenotype may be associated with a more aggressive cancer cells and metastatic cancer cells. However, the increased Axl and CD24 expression may enhance the susceptibility of the cancer cells to lysis induced by Zika virus infection and combat the increased metastasis, growth, and invasiveness of the cancer cells.

Similarly, TYRO3 and MERTK are also known to be involved in the processes of immune regulation, platelet aggregation, and cell proliferation, survival, and migration. Thus, increased expression of these receptors in tumors may cause increased metastasis and growth of tumor (or cancer) cells. MDA5 has been found to increase growth arrest and cell death when expressed in cancer cells. Additionally, expression of TLR3 may be a marker and/or associated with tumor cell metastasis in certain types of cancer. It has additionally been found that TLR8 expression in tumor cells may cause increased tumor growth. TIM1 has been found to be involved in tumor invasion and metastasis and may be related to tumor development and TIM4 has been identified as promoting lung cancer cell growth and proliferation when it is overexpressed. For at least the above recited reasons, targeting of these receptor proteins through the treatment with Zika virus may reduce cancer cell metastasis, tumor growth, and various other characteristics of expression of these receptors.

Subjects that can receive a treatment according to the present disclosure generally include any patient diagnosed with a tumor characterized as CD24 positive (CD24⁺) tumor. While the treatment and examples will be described herein with reference to primarily ovarian cancer, the CD24 positive tumor may be selected from the group consisting of: an ovarian cancer, a colorectal cancer, a B cell lymphoma, erythroleukemia, a glioma, a non-small cell lung cancer, an esophageal squamous cell carcinoma, a hepatocellular carcinoma, a hepatoblastoma, a cholangiocarcinoma, a pancreatic adenocarcinoma, a melanoma, a testicular tumor, an urothelial carcinoma, a breast cancer, a primary neuroendocrine carcinoma, a neural crest-derived tumor (e.g., a neuroblastoma), a human papillomavirus (HPV)-associated malignancy, an Epstein-Barr virus-induced malignancy, and a prostate carcinoma. The HPV-associated malignancy may be selected from the group consisting of cervical cancer or precancer, vaginal, vulvar, and anal precancers or cancers, and oropharyngeal precancers or cancers. An Epstein-Barr virus-induced malignancy may be selected from the group consisting of nasopharyngeal carcinoma, lymphoma, and post transplantation lymphoma proliferative disease.

Patients harboring CD24-positive tumors or who are at risk of developing such tumors are selected for Zika virus therapy on the basis of CD24 cells populating the tumor mass. Tumor cells may be assessed for the presence of CD24 using common histopathology or PCR-based methods.

A given tumor may contain a mixture of both CD24-positive and CD24-negative cells. Such tumors will also benefit from Zika virus oncolytic treatment since the eradication of Zika-sensitive CD24 positive tumor cell may lead to tumor shrinkage and/or reduction in growth, thus resulting in some therapeutic benefit to the patient. Furthermore, CD24-negative tumors may express Axl or other putative Zika virus receptor proteins (e.g., DC-SIGN, Tyro3, TIM1, TIM4, TLR3, TLR8, RIG-1, MDA5, Mer-TK) that permit Zika virus infection of tumor cells. Accordingly, Zika virus oncolytic treatment may be carried out on tumors comprising both CD24⁺ and CD24⁻ cells, although greater anti-tumor response would occur in tumors comprising predominately CD24⁺ tumor cells.

Subjects that can receive a treatment according to the present disclosure may also include any patient diagnosed with a tumor characterized as an Axl positive tumor. Even further, subjects may also include any patient diagnosed with DC-SIGN, Tyro3, TIM1, TIM4, TLR3, TLR8, RIG-1, MDA5, or Mer-TK positive tumors. As previously discussed, while the treatment and examples will be described herein with reference to primary ovarian cancer, the tumor may be selected from the group consisting of: colon cancer, breast cancer, lung cancer, liver cancer, thyroid cancer, melanoma, schwannoma, ovarian cancer, prostate cancer, leiomyosarcoma, dedifferentiated liposarcoma, undifferentiated pleomorphic sarcoma, synovial sarcoma, esophageal cancer, endometrial cancer, multiple myeloma, and several leukemia subtypes. However, various other diseases may be targeted, and the above list is provided as example.

Zika virus has a positive-sense, single-stranded RNA genome approximately 11 kilobases (kb) in length. The genome contains 5′ and 3′ untranslated regions flanking a single open reading frame that encodes a polyprotein that is cleaved into three structural proteins: the capsid (C), pre-membrane/membrane (prM), and envelope (E), and seven non-structural proteins (NS1, NS2A, NS2B, NS3, NS4A, 2K, NS4B, and NS5). Zika virus for use is an “oncolytic virus”, that is, it is a virus that preferentially replicates in, and kills, neoplastic cells. Delivery of the oncolytic Zika virus to a neoplasm can result in substantial lysis of the neoplastic cells infected by the virus. The term “substantial lysis” means at least about 10% of the cells of a neoplasm are lysed. More preferably, at least about 20%, 30%, 40%, 50%, 60%, 70%, 80% or 90% of the cells are lysed. Most preferably, at least about 95% of the cells are lysed. The percentage of tumor cell lysis can be determined, for example, by measuring the reduction in the size of the tumor or reduction of symptoms of the tumor.

Zika virus for administration may comprise naturally occurring (wild-type) Zika virus or modified Zika virus, or purified Zika virus RNA or its derivatives. The virus is “naturally occurring” when it can be isolated from a source (natural vector for the virus or an infected subject such as a human) in nature (or has been previously isolated from a natural source and stored in a biological depository).

A “modified” Zika virus is a Zika virus other than a naturally-occurring Zika virus. Accordingly, the term “Zika virus” as used herein refers to both a naturally occurring and a modified virus, and encompass both Zika virus particles and naked Zika virus RNA.

The modified Zika virus is still capable of lytically infecting a target neoplastic cell of a host subject. For example, the genetic material of the virus may be mutated, or the virus particle may be modified. The modified virus may be a recombinant virus, for example a virus engineered to express a heterologous protein.

The virus may be modified by incorporation of mutated coat proteins, such as for example, into the virion capsid. The purpose of such an intentional mutation would be to allow repeat Zika virus administration by avoiding immune system virus neutralization as would occur with initial zika virus infection (e.g., Zika virus protein epitopes found to be targets of immune system-induced neutralization would be genetically modified to avoid immune system recognition and consequent neutralization. Viral protein mutation might also be used to optimize tumor cell-specific virus-induced lytic activity). The proteins may be mutated by replacement, insertion, or deletion. Replacement includes the insertion of different amino acids in place of the native amino acids. Insertions include the insertion of additional amino acid residues into the protein at one or more locations. Deletions include deletions of one or more amino acid residues in the protein. Such mutations may be generated by methods known in the art. For example, oligonucleotide site-directed mutagenesis of the gene encoding for one of the coat proteins could result in the generation of the desired mutant coat protein. Expression of the mutated protein in virus infected mammalian cells in vitro such as COS 1 cells or Vero cells will result in the incorporation of the mutated protein into the virus virion particle.

The virus may be modified to reduce or eliminate an immune reaction to the virus. Such a modified virus is termed an “immunoprotected virus”. Such modifications could include packaging of the virus in a liposome, a micelle, or other vehicle to mask the virus from the host immune system. For example, the virion may be treated with chymotrypsin in the presence of micelle-forming concentrations of alkyl sulfate detergents to generate a new infectious subvirion particle. Alternatively, the outer capsid of the virion particle may be removed since the proteins present in the outer capsid are the major determinant of the host humoral and cellular responses.

The virus may be a recombinant virus resulting from the recombination/reassortment of genomic segments from two or more genetically distinct viruses. Recombination/reassortment of virus genomic segments may occur in nature following infection of a host organism with at least two genetically distinct viruses. Recombinant virions can also be generated in cell culture, for example, by co-infection of permissive host cells with genetically distinct viruses.

It is preferable to avoid immune responses against the virus, particularly in animals that have previously received large amounts of the same virus or closely-related virus. Immune responses may be avoided if the virus is of a subtype or strain to which the mammal has not developed immunity or has not been vaccinated against.

While genetic modifications of the oncolytic Zika virus (that do not result in loss of oncolytic activity) may be carried out, modification may not be required from a safety standpoint. For example, the oncolytic Zika virus strains MR766 and PRVABC59 (ATCC© VR-1843), MR766, as well as the Nicaragua and Brazil strains being the strain utilized in the Examples, has no intended genetic modifications, although the virus has been passaged through and maintained in Vero cells, and as a consequence, may differ in RNA sequence from the originally-isolated virus. While longitudinal studies are ongoing to determine whether or not Zika virus infection in children and adults causes some subtle brain injury, no definitive findings have been reported. Zika virus infection in children and adults are asymptomatic in roughly 80% of individuals. Symptomatic cases or most often mild cases present with fever, rash, and conjunctivitis.

While attenuated Zika viruses may be considered for therapeutic use, the fact that 80% of Zika virus infections are asymptomatic, and the remaining 20% of infections are generally very mild, as well as the rarity of severe Zika virus disease, attenuation of Zika viruses for therapy may be unnecessary. Moreover, since the anti-cancer effect of Zika virus as demonstrated herein relies on virus-mediated lysis of target tumor cells, attenuation may adversely hinder the therapeutic effect of the virus. In general, genetically modified Flaviviruses are unstable and frequently nonfunctional, suggesting that native Zika viruses may be more suitable for therapeutic use. In addition, attenuation may impair the ability of the virus to replicate and cause viremia, phenomena that underly the ability of the virus to infection tumors.

Any suitable source of the oncolytic Zika virus may be used provided it can be prepared according to the Food and Drug Administration GMP standards (other the standards of other relevant regulatory agencies). The oncolytic activity of a candidate Zika strain for use may be determined by appropriate oncolytic assay, as described in the Examples that follow. For example, ovarian cancer cell lines including SkOV3, ES2, A2780, or a panel including other cell lines, is infected with the candidate Zika virus strain for oncolytic therapy, and cell toxicity can be determined by an appropriate proliferation assays. One such assay, known as the CellTox™ Green assay, is a real-time assay using a fluorescent DNA binding dye to monitor the number of viable cells in the assay. The test relies on the ability for the fluorescent DNA binding dye to bind with DNA released from cells after the loss of membrane integrity, and the inability for the dye to enter live cells. One such cytotoxicity assay is available from Promego Corp., Madison, WI as the CellTox™ Green assay.

Wild-type Zika strains that may be screened for oncolytic activity in this manner include, for example, the following strains commercially available from the American Type Culture Collection, 10801 University Boulevard, Manassas, Virginia, 20110: MR 766 (ATCC® VR84), originally isolated by Dick et al, Trans R Soc Trop Med Hyg 46: 509-520, 1952; and an MR 766 tissue culture-adapted strain from ATCC® VR-84 (ATCC® VR-1838). The following Zika strains commercially available from BEI Resources, 10801 University Boulevard, Manassas, VA, 20110, a depository established by the National Institute of Allergy and Infectious Diseases (NIAID) and managed by ATCC, may likewise be screened for oncolytic activity: MR 766 (NR-50065); IbH 30656 (NR-50066); FLR (NR-50183); H/PAN/2016/BEI-259634 (NR-50210); H/PAN/2015/CDC-259359 (NR-50219); H/PAN/2015/CDC-259249 (NR-50220); H/PAN/2015/CDC-259364 (NR-50221); PRVABC59 (NR-50240); P 6-740 (NR-50245); MEX I-44 (NR-50279); DAK AR 41524 (NR-50338); R103451 (NR-50355); PLCal_ZV (NR-50234); MEX 2-81 (NR-50280); MEX I-7 (NR-50281); and Nicaragua (ZIKV-Nicaragua/2016 (UCB 7420)) and Brazil (ZIKV-SJRP/2016 (184)). The candidate virus may be one or more isolates from one or more species, including but not limited to avian, insect and mammalian species. An oncolytic Zika virus may be modified as described above to provide a modified Zika virus for administration, or the native unmodified virus may be administered.

PRVABC59 (ATCC® VR-1843) is an oncolytic Zika virus strain that can be used. It is an Asian-lineage Zika virus. Virus strains circulating in the Caribbean, and in Central and South America are Asian-lineage Zika viruses. Reports suggesting that African-lineage Zika virus strains are less neurotoxic than Asian-lineage Zika virus strains. Accordingly, Asian-lineage Zika virus strains may be preferred, as they are likely to have increased lytic action in comparison to African-lineage Zika viruses. However, the oncolytic Zika virus strain selected for a patient should not be the same strain used in any eventual Zika virus vaccine administered to the patient, which could impact the oncolytic ability of that virus. Furthermore, the strain of Zika virus optimal for therapeutic use may vary bother by tumor type and host patient.

The oncolytic Zika virus may be administered to the subject in its native state, i.e., virus particles. Alternatively, the virus may be administered as naked viral nucleic acid, e.g., viral RNA, encoding the virus. Accordingly, unless indicated otherwise, “Zika virus” as used herein means either the native virus in the form of virus particles or naked RNA encoding a Zika virus. It should be appreciated that by “naked” viral RNA means RNA without the associated proteins that comprise a virus particle. Thus, “naked” viral RNA does not exclude RNA combined with formulation agents, such as liposome-forming agents, or other pharmaceutical vehicles. Naked viral RNA may be isolated from viral particles by known techniques or may be made synthetically.

The naked viral RNA may be administered in the form of liposomes. Liposomes are generally derived from phospholipids or other lipid substances and are formed by mono- or multi-lamellar hydrated liquid crystals that are dispersed in an aqueous medium. Any non-toxic, physiologically acceptable and metabolizable lipid capable of forming liposomes can be used. Compositions in liposome form may contain stabilizers, preservatives, excipients, and the like. The preferred lipids are the phospholipids and the phosphatidyl cholines (lecithins), both natural and synthetic. For example, liposomes may be artificially formed with the intent of being used to deliver the naked viral RNA. Methods for forming liposomes are known in the art. See, Prescott, Ed., Methods in Cell Biology, Volume XIV, Academic Press, New York, N.Y. (1976), p. 33 et seq. In one exemplary embodiment, liposomes are formed with the cationic liposome formulation Lipofectamine™ 2000. Viral RNA: Lipofectamine™ 2000 complexes (vRNA:lipid) may be prepared according to manufacturer's instructions (Thermo Fisher Scientific Inc.). Briefly, various amounts of vRNA and 2 μl Lipofectamine™ 2000 can be diluted separately in 50 μl serum and antibiotic free media and gently mixed. Five minutes following Lipofectamine™ 2000 dilution, the corresponding vRNA and lipid solutions can be combined and gently mixed. The mixture is incubated at room temperature for 45 minutes to allow vRNA:lipid complexes to form. Once formed, the complexes can be administered to the subject in need of cancer treatment for a CD24⁺ tumor.

The therapeutic agent may comprise a single species of oncolytic Zika viruses. Alternatively, a combination of at least two oncolytic Zika viruses may be administered. The viruses may be administered at the same time, by the same route, or at different times, by different routes.

The oncolytic Zika virus is administered to a subject in a manner that results in contact of the virus with target neoplastic cells. The route by which the virus is administered, as well as the formulation, carrier or vehicle, will depend on the location as well as the type of the neoplasm. A wide variety of administration routes can be employed. For example, the virus may be administered intradermally. Intradermal virus injection leads to local viral replication in the skin. Ultimately, local injection leads to viral replication with consequent systemic infection with viremia. The viremia (i.e., hematogenous spread of virus) would lead to infection to any tumor receiving blood supply. Data from human and animal studies indicate that systemic Zika virus infection results in virus crossing the blood-brain barrier. It is expected that the virus will also cross the blood-testis and blood-eye barrier. For a solid neoplasm that is accessible, the virus can be administered by injection directly to the neoplasm. For neoplasms that are not easily accessible within the body, such as metastases or brain tumors, the virus can be administered intravenously such that it can be transported systemically through the body of the mammal and thereby reach the neoplasm (e.g., intravenously, or intramuscularly). Finally, for treatment of malignancies in the brain, spinal cord, or eye, virus may be administered into the brain ventricles, intrathecally into the cerebrospinal fluid, or intraocularly into the anterior or posterior chambers of the eye, providing direct assess of virus to tumor(s) in the brain, spinal cord, or eye.

While the disclosure herein is described largely with reference to the use of Zika virus, any variety of flaviviruses may be used for these purposes. In some instances, among other flaviviruses, a flavivirus having a similar lineage to that of Zika virus may be used. A flavivirus having a similar lineage may be defined as having homologies in the RNA sequence. For example, the RNA sequence of the viruses may be substantially the same or may have at least 60% identical RNA sequencing. In some instances, approximately 75% of the RNA sequence of a similar virus may be identical to approximately 75% of the RNA sequence of Zika virus. Further, two or more flaviviruses may be combined and utilized as a treatment.

For example, Spondweni virus may be explored for use in treated diseases that cause cells to express CD24 and/or Axl or other putative Zika virus receptor proteins. The Spondweni viruses have strong sequence homologies to Zika viruses. Spondweni viral infections in humans cause a non-specific febrile illness and most cases in humans are asymptomatic or minimally symptomatic. Further, human teratogenicity of Spondweni virus has not been reported. For these reasons, it is believed that Spondweni virus may also be used as an oncolytic treatment for CD24-positive and/or Axl positive human cancers.

Additionally, the Kedougou virus is believed to have effectiveness as an oncolytic treatment for CD24-positive and/or Axl positive human cancers. Kedougou virus has a strong sequence homology to Zika virus. Further, it is believed that Kedougou virus likely causes mild illness in humans. For these reasons, it is believed that Kedougou virus may be used as an oncolytic treatment for CD24-positive and/or Axl-positive human cancers.

Because cancers such as ovarian cancers may be metastatic to many sites, local/intratumor administration may or may not be effective. Zika virus may be administered intradermally, subcutaneously, or intravenously, with the anticipation that the patient will become transiently viremic, with the consequent hematogenous delivery of virus to the tumor. In such cases, viruses that are administered systemically, i.e., by intravenous injection, will spread to the locations of the neoplastic cells, resulting in lysis of the cells.

Alternatively, the virus can be administered directly to a single solid neoplasm, where it then is carried systemically through the body to metastases. The virus can also be administered topically, e.g., for melanoma. The virus can be administered systemically to mammals which are immune compromised, or which have not developed immunity to the virus. More than one route of administration may be used to deliver the oncolytic Zika virus.

In certain embodiments, the oncolytic virus is delivered by direct injection to a tumor (e.g., intralesional injection), or by systemic administration. Intralesional injection of a tumor may be performed by any appropriate means known to the skilled person, taking into account factors such as the type of tumor being treated, the size and location of the tumor, accessibility of the tumor to direct injection. Injection techniques that increase or maximize the distribution of the virus throughout the tumor may offer improved therapeutic outcomes. For example, in the treatment of solid tumors, multiple lesions may be injected in a dose hyper-fraction pattern, starting with the largest lesion(s) (2.0 mL injected into tumors >2.5 cm, 1.0 mL into 1.5 to 2.5 cm; 0.5 mL into 0.5 to 1.5 cm) to a 4.0 mL maximum.

Direct administration to the brain (or to a specific region of the brain) may be achieved, for example, and not by way of limitation, by local infusion (e.g., during surgery), by injection (e.g., intracerebroventricular injection), by means of a catheter, or by means of a ventricular reservoir or pump placed in the tumor cavity during surgery or implanted subcutaneously in the scalp and connected to the brain via an outlet catheter. Alternatively or additionally, local administration can be achieved via the use of an implant device (e.g., a wafer implant containing the active ingredient) or a drug depot that can be placed locally during surgery. Such systems provide sustained oncolytic virus release.

Since various types of cancers, such as ovarian cancer, can be widely metastatic, intratumor injection is not likely to be an effective therapy for most patients, unless there is a subsequent systemic dissemination of virus.

Before, during or after the administration of the oncolytic Zika virus, the subject may be given an immunosuppressive therapy to facilitate or enhance the effect of the virus treatment. The immunocompetency of the subject of the oncolytic Zika virus treatment may be suppressed by an immunosuppressive therapy comprising the co-administration (or prior or subsequent) administration of pharmaceuticals known in the art to suppress the immune system in general or alternatively by administration of anti-idiotypic antibodies that recognize the antibodies for the administered virus.

The oncolytic Zika virus may be administered to immunocompetent mammals in conjunction with the administration of immunosuppressants and/or immunoinhibitors. Such immunosuppressants and immunoinhibitors are known to those of skill in the art and include but are not limited to such agents as cyclosporin, rapamycin, tacrolimus, mycophenolic acid, azathioprine and their analogs, and the like. Other agents are known to have immunosuppressant properties as well (see, e.g., GOODMAN AND GILMAN, 7th Edition, page 1242).

Such immunoinhibitors also include anti-antivirus antibodies, which are antibodies directed against anti-virus antibodies that specifically recognize the virus of interest. Such antibodies can be made by methods known in the art. See for example Antibodies: A laboratory Manual, E. Harlow and D. Lane, Cold Spring Harbor Laboratory, 1988). Such anti-antivirus antibodies may be administered prior to, at the same time, or shortly after the administration of the virus. Preferably an effective amount of the anti-antivirus antibodies are administered in sufficient time to reduce or eliminate an immune response by the mammal to the administered virus.

The humoral immunity of the mammal against virus may also be temporarily reduced or suppressed by plasmapheresis of the mammal's blood to remove antibodies specific for that virus. The humoral immunity of the mammal against the virus may additionally be temporarily reduced or suppressed by the intravenous administration of non-specific immunoglobulin to the mammal. The immune system may also be suppressed by anti-CD4 and/or anti-CD8 antibodies or complement neutralization.

The anti-tumor effect of Zika viruses may also be increased by driving target tumor cells into a state of autophagy. Cells undergoing autophagy show increased susceptibility to Zika virus-induced lysis. Methods for inducing cellular autophagy include but are not limited to cis-platinum and its derivatives, carboplatin, doxorubicin, mTOR inhibitors (e.g., rapamycin, rapalogues, torin1, dactolisib), agents that inhibit mTOR by activation of adenosine monophosphate-activated protein kinase (e.g., metformin, trehalose, resveratrol), statins (e.g., simvastatin, atorvastatin), and ionizing radiation.

Zika viruses may be grown to high titer in Vero cells. Vero cells are a commercially available immortal cell like derived from the kidney of an African Green Monkey. Vero cells are commonly used for the expansion of viruses. Zika virions may be purified by ultrafiltration and/or by gradient sedimentation, according to known techniques.

The oncolytic Zika virus may be purified for therapeutic administration by standard methodology. As used herein, “purified viruses” refer to viruses that have been separated from cellular components that naturally accompany them. Typically, viruses are considered purified when they are at least 70% (e.g., at least 75%, 80%, 85%, 90%, 95%, or 99%) by dry weight, free from the proteins and other cellular components with which they may be naturally associated.

There are several advantages to using purified Zika virus RNA for therapeutic administration through transfection. Viral RNA is stable and thus is not prone to mutation, adaptation or drift which may otherwise occur when Zika virus is administered through cell lines. Further, while the viral RNA produce is large (10.7 kb), it can be synthesized chemically. This may allow for the incorporation of modified bases to increase the stability and expression of Zika virus. Further stabilized or optimized Zika virus RNA may be prepared using modified RNA bases. Additionally, rather than chemical synthesis, a cDNA for Zika virus may be made. From this cDNA template, an RNA viral genome may be prepared. Due to the stability of cDNA, transcription from cDNA provides RNA production for a highly stable sequence template with consequent viral RNA sequence consistency. Further, modification of cDNA allows for modification of the infecting RNA. Additionally, viral RNA doses are readily quantified, and it may be safer to administer viral RNA since RNA is not infectious without an appropriate transfection reagent.

The oncolytic Zika virus may be formulated appropriately for pharmaceutical administration. Pharmaceutical compositions comprise one or more oncolytic Zika viruses and a pharmaceutically acceptable carrier. A pharmaceutically acceptable carrier can be a solid, semi-solid, or liquid material that can act as a vehicle, carrier or medium for the virus. For infusion, particularly intravenous infusion, the virus may be formulated in an aqueous solution of mineral salts or other water-soluble molecules, e.g., normal saline, a solution of sodium chloride at 0.9% concentration; Ringer's lactate or Ringer's acetate; or a solution of 5% dextrose in water (“D5W”). Pharmaceutical compositions can be formulated to provide quick, sustained or delayed release of a virus after administration by employing procedures known in the art. Various other formulations for use in a pharmaceutical composition can be found in Remington, The Science and Practice of Pharmacy 22nd ed., Loyd V. Allen et al, editors, Pharmaceutical Press (2013). The oncolytic Zika virus may be administered topically, particularly for the treatment of melanoma, in the form of a gel, ointment or other semi-solid formulation, for example. The virus may be contained in a transdermal device to provide continuous or discontinuous virus delivery. The construction and use of transdermal patches for the delivery of pharmaceutical agents is known in the art. See, for example, U.S. Pat. No. 5,023,252. Such patches can be constructed for continuous, pulsatile, or on-demand delivery of viruses.

The oncolytic Zika virus is administered to a subject in need of such treatment in an amount that is sufficient to treat the neoplasm. A neoplasm is treated when administration of virus to the proliferating cells effects lysis of the proliferating cells. This may result in a reduction in size of the neoplasm or a complete elimination of the neoplasm. The reduction in size of the neoplasm, or elimination of the neoplasm, is generally caused by lysis of neoplastic cells by the virus. Preferably the effective amount is that amount able to at least inhibit tumor cell growth. Preferably the effective amount is from about 1 pfu/kg body weight to about 10¹⁵ pfu/kg body weight, and more preferably from about 100 pfu/kg body weight to about 10¹³ pfu/kg body weight. For example, for treatment of a human subject, approximately 100 to 10¹⁷ pfU of virus can be used, depending on the type, size, location and number of tumors present. The effective amount will be determined on an individual basis and may be based, at least in part, on consideration of the type of virus; the chosen route of administration; the individual's size, age, gender; the severity of the patient's symptoms; the size and location or other characteristics of the neoplasm; and the like. Given that Zika viruses are transmitted in nature by mosquitos at very low concentration, dosages of less than 200 pfU may be possible. However, effective dosages may have a value ranging between approximately 200 pfU and approximately 100,000,000 pfU. As previously described, these dosages may be given in multiple administrations or in a single dose.

Treatment efficacy may be assessed both by tumor size and based upon biochemical markers, such as homovanillic acid (HVA) and vanillylmandelic acid (VMA), cancer antigen 125 (CA125) and human epididymis protein 4 (HE4). Elevated values of HVA, and VMA, and other catecholamine metabolites, are markers for the presence of suggestive of the presence of neural crest tumors, particularly neuroblastoma. CA125 and HE4 are markers of the presence of ovarian cancer.

Because of the likely development of humoral immunity, an initial course of treatment an initial treatment course is likely to be limited to, e.g., five days in the absence of steps taken temporarily reduce or suppress patient humoral immunity.

The compositions are preferably formulated in a unit dosage form, each dosage containing from about 10² pfu to about 10⁸ pfu of virus. The term “unit dosage forms” refers to physically discrete units suitable as unitary dosages for human subjects and other mammals, each unit containing a predetermined quantity of virus calculated to produce the desired therapeutic effect, in association with a suitable pharmaceutical excipient.

A stock of the oncolytic Zika virus composition may be diluted to an appropriate volume suitable for dosing, for example to achieve the desired dose of viral particles administered in a desired volume. The volume in which the virus is administered will be influenced by the manner of administration. For example, intravenous administration of virus may typically use about 5 ml to about 500 ml of virus diluted in normal saline, infused by an automatic pump over approximately 30 minutes. Subcutaneous injection might entail administration of 0.25 to 2 mL.

Dosages of oncolytic Zika virus vary and are administered in one or more dose administrations, for example, daily, for one or several days. The virus is administered in a single dose or in multiple doses (e.g., two, three, four, six, or more doses). The multiple doses can be administered concurrently, or consecutively. The virus can also be administered to more than one neoplasm in the same individual. The virus may be administered, for example, continuously to a subject at least once per day or up to intermittently or continuously throughout the day on consecutive days, for a period of time.

Because the patient will become viremic in response to Zika virus administration, the patient will mount a humoral and cellular response that is likely to be neutralizing, within 10-21 days of first administration. Such a virus-neutralizing response will render further virus administrations ineffective, unless Zika viruses with capsids that express divergent epitopes are used in subsequent administrations, if another closely related virus (e.g., Spondweni virus, Kedougou virus, or another flavivirus with oncolytic properties) or steps are taken to reduce or suppress the patient immune response.

In certain embodiments, the virus is administered, for example, to subjects by means of intravenous administration in any pharmacologically acceptable solution, or as an infusion over a period of time. For example, the substance may be administered systemically by injection, or administered by infusion in a manner that results in the daily delivery into the tissue or blood stream of the subject. Where the administration is by infusion, the infusion can be a single sustained dose or can be delivered by multiple infusions.

The oncolytic Zika virus-based treatment may be combined with other anti-neoplasm treatment modalities, such as chemotherapy (with another chemotherapeutic agent active agent other than an oncolytic Zika virus), radiotherapy, surgery, hormone therapy and/or immunotherapy. For example, the virus may be administered in conjunction with surgery or removal of the neoplasm. Administration of virus at or near to the site of the neoplasm can be combined with surgical removal. The oncolytic Zika virus may be administered in conjunction with or in addition to radiation therapy. The oncolytic Zika virus may be administered in conjunction with or in addition to one or more anti-cancer agents, also known as “chemotherapeutic agents”. These terms refer to those medications that are used to treat cancer or cancerous conditions. Anti-cancer drugs are conventionally classified in one of the following group: alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, anti-hormones and anti-androgens. Examples of such anti-cancer agents include, but are not limited to, BCNU, cisplatin, carboplatin, gemcitabine, hydroxyurea, taxanes (e.g., docetaxel, paclitaxel), temozomide, topotecan, fluorouracil, vincristine, vinblastine, procarbazine, decarbazine, altretamine, methotrexate, mercaptopurine, thioguanine, fludarabine phosphate, cladribine, pentostatin, cytarabine, azacitidine, etoposide, teniposide, irinotecan, docetaxel, doxorubicin, daunorubicin, dactinomycin, idarubicin, plicamycin, mitomycin, bleomysin, tamoxifen, flutamide, leuprolide, goserelin, aminogluthimide, anastrozole, amsacrine, asparaginase, mitoxantrone, mitotane, and amifostine.

Methods disclosed herein may also be employed together with one or more further combinations of cytotoxic agents as part of a treatment regimen, wherein the further combination of cytotoxic agents is selected from: CHOPP (cyclophosphamide, doxorubicin, vincristine, prednisone, and procarbazine); CHOP (cyclophosphamide, doxorubicin, vincristine, and prednisone); COP (cyclophosphamide, vincristine, and prednisone); CAP-BOP (cyclophosphamide, doxorubicin, procarbazine, bleomycin, vincristine, and prednisone); m-BACOD (methotrexate, bleomycin, doxorubicin, cyclophosphamide, vincristine, dexamethasone, and leucovorin); ProMACE-MOPP (prednisone, methotrexate, doxorubicin, cyclophosphamide, etoposide, leucovorin, mechloethamine, vincristine, prednisone, and procarbazine); ProMACE-CytaBOM (prednisone, methotrexate, doxorubicin, cyclophosphamide, etoposide, leucovorin, cytarabine, bleomycin, and vincristine); MACOP-B (methotrexate, doxorubicin, cyclophosphamide, vincristine, prednisone, bleomycin, and leucovorin); MOPP (mechloethamine, vincristine, prednisone, and procarbazine); ABVD (adriamycin/doxorubicin, bleomycin, vinblastine, and dacarbazine); MOPP (mechloethamine, vincristine, prednisone and procarbazine) alternating with ABV (adriamycin/doxorubicin, bleomycin, and vinblastine); MOPP (mechloethamine, vincristine, prednisone, and procarbazine) alternating with ABVD (adriamycin/doxorubicin, bleomycin, vinblastine, and dacarbazine); ChlVPP (chlorambucil, vinblastine, procarbazine, and prednisone); IMVP-16 (ifosfamide, methotrexate, and etoposide); MIME (methyl-gag, ifosfamide, methotrexate, and etoposide); DHAP (dexamethasone, high-dose cytaribine, and cisplatin); ESHAP (etoposide, methylpredisolone, high-dose cytarabine, and cisplatin); CEPP(B) (cyclophosphamide, etoposide, procarbazine, prednisone, and bleomycin); CAMP (lomustine, mitoxantrone, cytarabine, and prednisone); CVP-1 (cyclophosphamide, vincristine, and prednisone), ESHOP (etoposide, methylpredisolone, high-dose cytarabine, vincristine and cisplatin); EPOCH (etoposide, vincristine, and doxorubicin for 96 hours with bolus doses of cyclophosphamide and oral prednisone), ICE (ifosfamide, cyclophosphamide, and etoposide), CEPP(B) (cyclophosphamide, etoposide, procarbazine, prednisone, and bleomycin), CHOP-B (cyclophosphamide, doxorubicin, vincristine, prednisone, and bleomycin), CEPP-B (cyclophosphamide, etoposide, procarbazine, and bleomycin), and P/DOCE (epirubicin or doxorubicin, vincristine, cyclophosphamide, and prednisone).

As will be appreciated by one skilled in the art, the selection of one or more therapeutic agents to be administered in combination with a method of treatment of the present disclosure will depend on the tumor to be treated.

Combinations of the oncolytic Zika virus and chemotherapeutic agents are administered either concomitantly (e.g., as an admixture), separately but simultaneously (e.g., via separate intravenous lines into the same subject), or sequentially (e.g., one of the compounds or agents is given first followed by the second). Thus, the term “combination” is used to refer to concomitant, simultaneous, or sequential administration of two or more agents.

The concomitant administration of Zika virus and chemotherapy may provide several benefits during the treatment of cancer or other diseases characterized by CD4 and Axl expression. Chemotherapy tends to induce an increased expression of CD24 or CD24 and Axl. Therefore, the oncolytic activity of Zika virus would be enhanced due to this increase expression of CD24 and/or Axl as these both act as surface receptors for Zika virus, as will be described further herein. Thus, the continued treatment with chemotherapy may actually increase the lysis of the cancer cells during treatment with Zika virus. Additionally, if the cancer cells are able to mutate to reduce the expression of CD24 and/or Axl and avoid the oncolytic activity of Zika virus infection, the cells may then exhibit an increased susceptibility to chemotherapy. Thus, concomitant Zika virus and chemotherapy administration may be synergistic for tumor destruction and prevent or delay both chemotherapy resistance and Zika virus resistance of the cancer cells.

The interaction between a virus and its cellular receptor(s) is important for the determination of viral tissue and host tropisms. For instance, viral infection may be blocked by a viral-receptor binding protein. Accordingly, a viral cell receptor, and the viral molecule(s) that bind the receptor, are promising targets for developing anti-viral strategies. As demonstrated herein, human CD24, a cell surface glycoprotein, has been identified as a part of Zika virus host cell entry pathway. The identification of a cell surface receptor for Zika virus enables a focused strategy to, for instance, identify the CD24-binding molecule(s) of Zika virus. For instance, preparing anti-Zika virus antibodies that specifically block binding to CD24 provides a handle to identify Zika virus CD24-binding molecule(s). The viral CD24-binding molecule(s) is a candidate as an effective vaccine antigen. It is worth noting that Zika virus tends to mutate quickly, and viral pseudo species can develop during the course of infection. However, given the strong selective pressure presented by CD24 binding being a part of viral entry into a host cell, it is not unreasonable to expect that the portion of the viral molecule(s) that binds CD24 is less likely to mutate. Therefore, identifying the viral CD24-binding molecule is a promising avenue for obtaining effective vaccine antigens.

Additionally, developing anti-human CD24 antibodies that block Zika virus binding to CD24 are also useful. For instance, an anti-CD24 antibody (monoclonal antibodies or fragments thereof such as Fab, scFv, F(ab′)₂, and domain antibodies) that blocks Zika virus binding to CD24 is a candidate as prophylactic therapeutics for persons at risk of Zika virus exposure. An anti-CD24 antibody that blocks Zika virus binding to CD24 may also be useful in screening small molecule libraries for candidate molecules that block Zika virus binding to CD24. Such molecules are also candidates as prophylactic therapeutics for persons at risk of Zika virus exposure.

Further, Axl, a receptor tyrosine kinase often expressed by ovarian cancer cells, has been identified as a cell surface Zika virus receptor protein. This results in cells expressing higher levels of Axl as being candidates for interaction, and more particularly lysis, with Zika virus. As such, while described herein primarily in relation to tumors that are characterized as CD24-positive, the treatments and methods of treatments described herein may be applied with tumors that are characterized as Axl positive.

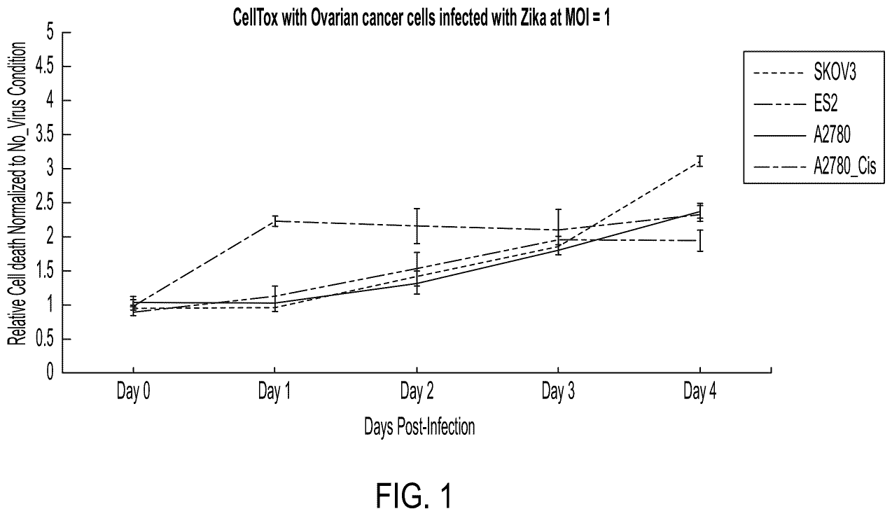

It has been found that the Nicaragua and Brazil strains in an ovarian cancel model are as effective as the MR766 virus at inhibiting the growth of ovarian tumors. Taken together, the co-expression of AXL and CD24 that occurs commonly in tumor cells but rarely occurs in somatic cells imparts two layers of specificity that allow Zika virus infection and productive replication in tumor cells while not supporting infection and replication in somatic cells (FIG. 1). Without being bound to a particular theory, it is believed that the co-expression of AXL and CD24 underlies the ability of Zika virus to infect and lyse RD cells effectively.

Additionally, as previously described throughout, various other Zika virus receptor proteins that may be expressed by tumors may be targeted in order to kill the tumor cells. For example, these Zika virus receptor proteins may include DC-SIGN, Tyro3, MER, TIM-1, TIM-4, TLR3, TLR8, RIG-1, MDA5 or other putative Zika virus receptor proteins. Any one of these receptor proteins may be expressed by a tumor independently or in combination with one another or with Axl and/or CD24. In this way, the methodology of using Zika virus to treat various tumors may be expanded to those that not only express CD24 or Axl, but any of the above receptor proteins. This may largely expand the number of diseases that can be treated with Zika virus and benefit from the Zika virus treatment and above described advantages of the treatment.

In result of the above description and the following examples, it is believed that Zika virus may have several uses in treatment of cancers. For example, a patient with progressive cancer due to the cells developing a resistance to the chemotherapy may be treated with Zika virus. Additionally, a patient may be treated by both a chemotherapy and Zika virus concomitantly to delay a patient's resistance to one or both of Zika virus and chemotherapy.

The practice of the disclosure is illustrated by the following non-limiting examples.

### Examples

### An Array of Ovarian Cancer Cell Lines are Permissive to Zika Virus Infection

Four human ovarian cancer cell lines were infected with Zika virus (strain=MR766) at a multiplicity of infection (MOI)=1. The ovarian cancer cell lines infected included SkOV3, ES2, A2780 and A2780_Cis. The cells were allowed to incubate for a total of four days, with samples acquired every one day (starting with Day 0). Acquired cells samples were screened for cell death and measured as fluorescence units and normalized to the uninfected condition (FIG. 1). The cell lysis was quantified through the use of by CellTox™ Green Cytotoxicity Assay.

The results indicate that each of the acquired and infected cell lines had a relative cell death normalized to noninfected cells that was above 1 after four days. More particularly the SkOV3 cells illustrated a relative cell death of approximately 3 after Day 4, while the ES2 cell line illustrated a relative cell death of approximately 1.75 after Day 4, and cell lines A2780 and A2780_Cis both exhibited a relative cell death of approximately 2.25 after Day 4. Overall, these results show that cultured SkOV3, ES2, A2780, and A2780-cis ovarian cancer cells are susceptible to Zika virus-induced lysis.

### Cytopathic Effect of Lytic Zika Virus Infection in Cultured Ovarian Cancer Cells

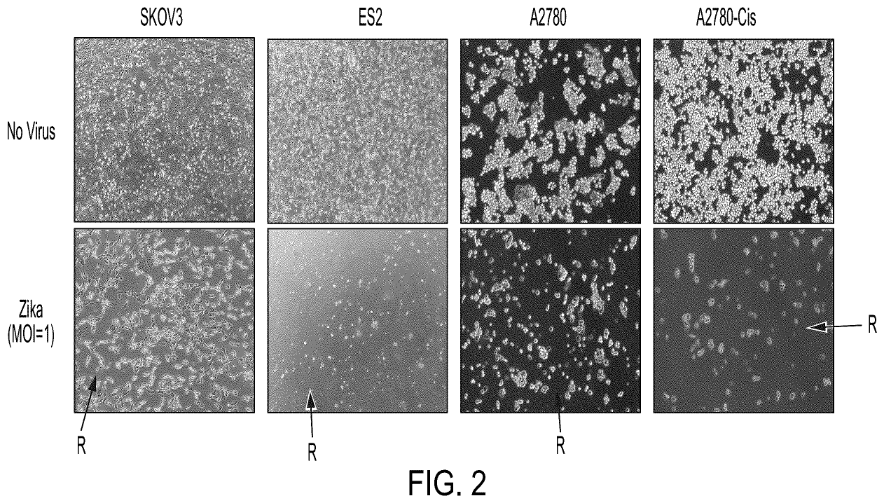

The cytopathic effect of lytic Zika virus infection in cultured ovarian cancer cells was examined in the next experiment. Four human cell lines of ovarian cancer, including SkOV3, ES2, A2780 and A2780_Cis, were infected with Zika virus (strain=MR766) at a multiplicity of infection (MOI)=1 and a sample of each cell line was also mock-infected with a control medium. At 72 hours after infection, a sample was gathered from each cell line, both infected with Zika virus and without Zika virus and imaged with Brightfield images at 40× magnification (FIG. 2). FIG. 2 illustrates that Zika virus infection causes rounding of the cells and subsequent lysis and detachment from the culture plate. The rounding of the cells is indicated by arrows R in the Zika virus infected cells. Further, the lysis and detachment of the cells in the Zika virus infected cells is illustrated in the reduced amount of cancer cells in the Zika virus infection samples as compared to the noninfected samples. These results indicate that cultured ovarian cancer cells infected with Zika virus are susceptible to Zika virus-induced lysis.

### Zika Virus-Infected Ovarian Cancer Cells Express Zika Virus-Encoded Envelope and NS1 Proteins

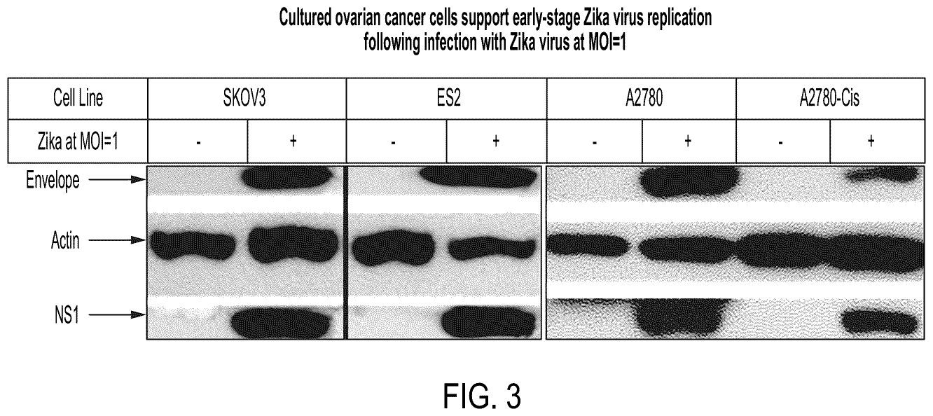

Early-stage Zika virus binding and replication within cultured ovarian cancer cells was examined. Zika virus binding to exposed cells was examined by demonstrating the presence of cell-associated viral envelope protein. Because the NS1 protein is not expressed until early in the course of infection, Zika virus infection was inferred by demonstration of de novo synthesis of Zika virus NS1 protein. In these experiments, four ovarian cancer cell lines (SkOV3, ES2, A2780, and A2780-cis) are shown after infection with a multiplicity of infection (MOI)=1 of Zika virus particles for two days. After two days of infection, the cells were harvested, lysed, and the extracts probed by Western blot analysis for expression of the viral NS-1 and envelope proteins. Beta-actin expression was measured as a loading control (FIG. 3).

Western blot analysis of the cell lines after infection revealed that all of the ovarian cancer cell lines tested showed evidence of the presence of Zika envelope protein (Env), confirming that virus had been introduced and attached to the cell surface. Further, the Western blot analysis of the cell lines after infection revealed that all of the ovarian cancer cell lines showed evidence of the presence of the NS-1 protein indicated that all cell lines were capable of virus uptake and expression of Zika virus proteins. Overall, these observations indicate that cultured ovarian cancer cells support early-stage Zika virus replication following Zika virus infection.

### Cis-Platinum Resistant and Doxorubicin-Resistant Ovarian Cancer Cells Susceptible to Zika Virus-Induced Lysis

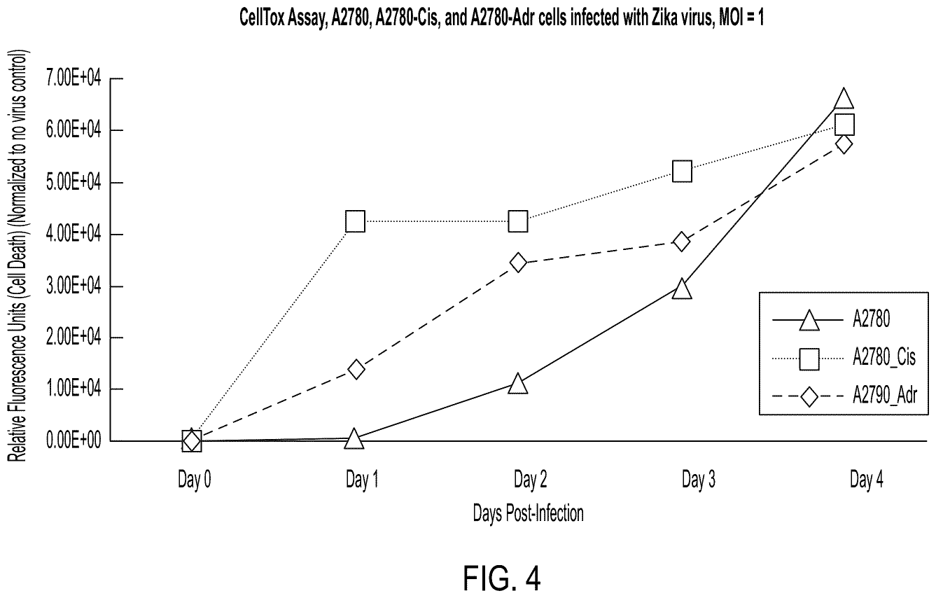

As previously discussed, cancer cells can often become resistance to various chemotherapies. The next experiment was conducted to study the susceptibility of chemotherapy resistant cancer cells to Zika virus-induced lysis in comparison to the susceptibility of chemotherapy sensitive cancer cells to Zika virus-induced lysis. In these experiments, the cis-platinum resistant cells (A2780-Cis), doxorubicin resistant cells (A2780-Adr), and cis-platinum and doxorubicin sensitive cells (A2780) were infected with multiplicity of infection (MOI)=1 Zika virus (strain=MR766). The cell lines were allowed to incubate for a total of four days, with infection occurring at Day 0 and with samples taken each one day. The cell death of each cell line was measured from each sample through CellTox™ Green assay (FIG. 4).

The results indicate that the cis-platinum resistant cells (A2780-Cis) and doxorubicin resistant cells (A2780-Adr) exhibited a greater and quicker susceptibility to Zika virus-induced lysis than the cis-platinum and doxorubicin sensitive cells (A2780) cells. For example, at Day 1, Day 2 and Day 3, the cis-platinum resistant cells (A2780-Cis) and doxorubicin resistant cells (A2780-Adr) exhibited greater relative cell death values than the relative cell death values exhibited by the cis-platinum and doxorubicin sensitive cells (A2780). Overall, the results indicate that cultured cis-platinum- and doxorubicin-resistant A2780 cells are lysed by Zika virus MR766 more rapidly than isogenic cis-platinum- and doxorubicin-resistant A2780 cells.

### Cis-Platinum Resistant and Doxorubicin-Resistant Ovarian Cancer Cells Susceptible to Zika Virus-Induced Lysis

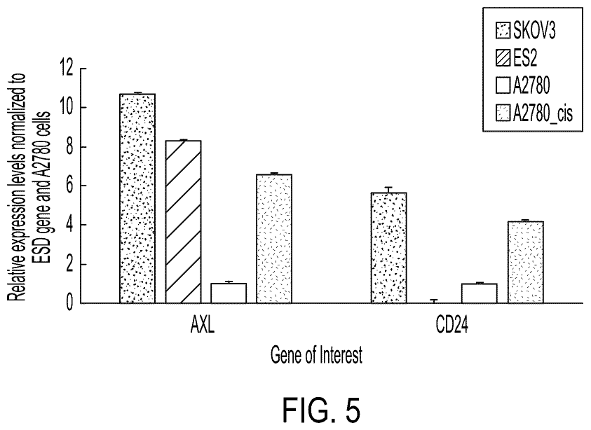

As a result of the previous experiment and the exhibition that the cis-platinum resistant cells (A2780-Cis) and doxorubicin resistant cells (A2780-Adr) exhibited a greater susceptibility to Zika virus-induced lysis than the cis-platinum and doxorubicin sensitive cells (A2780) cells, the next experiments explore the expression of Axl and CD24 mRNA in cis-platinum resistant cells (A2780-Cis), doxorubicin resistant cells (A2780-Adr), and cis-platinum and doxorubicin sensitive cells (A2780) cells. In these experiments, the real-time PCR analysis was performed on RNA gathered from various ovarian cancer cell lines (SkOV3, ES2, A2780, and A2780_cis). The RNA concentration was standardized. Esterase D (ESD) was used as the control gene to obtain the relative CD24 and Axl mRNA expression within each cell line. The mRNA expression for each cell line is showed in terms of fold change over ESD and then normalized to Axl and CD24 mRNA expression in A2780 cells (FIG. 5). The results indicate that that each of the ES2, SkOV3, and A2780_cis cell lines have a greater relative expression level of CD24 than the relative CD24 expression exhibited by the A2780 cells. Further, the results indicate that the cells of each of the SkOV3 and A2780_cis cell lines have a greater relative expression level of CD24 than the relative Axl expression exhibited by the cells of the A2780 cell lines.

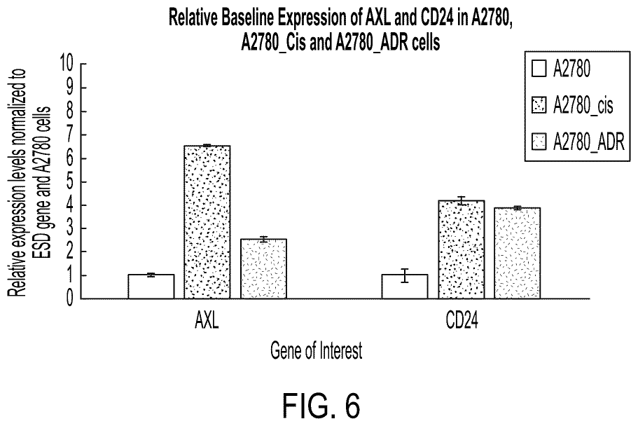

Further, the experiments also included conducting PCR analysis of cells gathered of the A2780 cell line, the A2780_cis cell line, and the A2780_ADR cell line. RNA was collected from each cell line, the RNA concentration was standardized for each cell line, and ESD was used as the control gene to obtain the relative CD24 and ACL mRNA expression. The relative expression of Axl and CD24 in each cell line is shown in FIG. 6. The mRNA expression is shown in terms of fold change over ESD and then normalized to Axl and CD24 expression in A2780 cells. The results indicate that cells from the A2780_cis and the A2780_ADR cell lines have higher relative expression levels of Axl and CD24 than the expression levels of Axl and CD24 exhibited by the A2780 cell line.

In Vivo Xenograft Treatment of SkOV3 Ovarian Cancer Tumors with Zika Virus in Mice

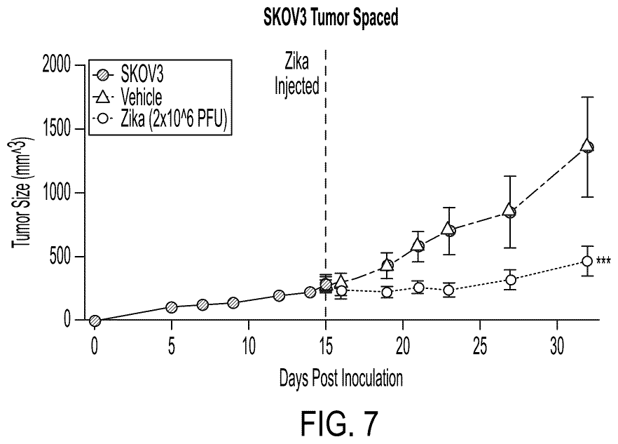

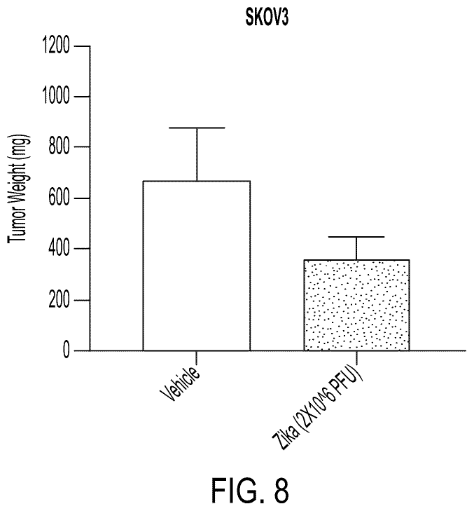

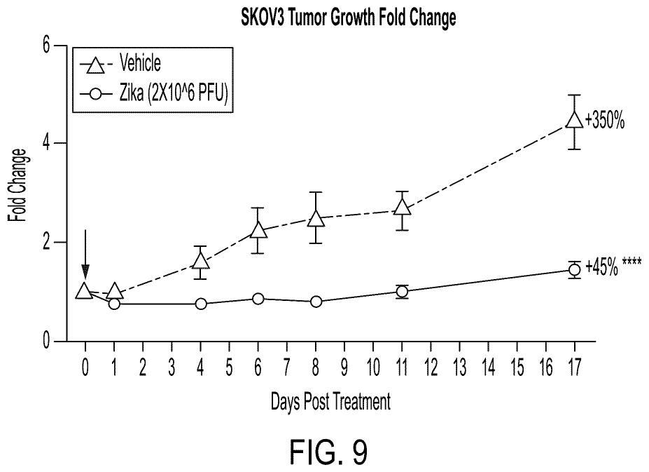

Further experiments were conducted in order to confirm the above results in vivo. In these experiments, subcutaneous xenograft tumors were established in female NSG (also Known As: NOD-scid IL2Rgamma^{null}, NOD-scid IL2Rg^{null}, or NOD scid gamma) mice over the course of twenty-nine days. The mice were obtained from the University of Central Florida Burnett School of Biological Sciences vivarium, which maintains an NSG mouse colony. The tumors are characterized by the SkOV3 ovarian cancer cell line. The sizes of the tumors within each of the mice were measured twice per week with Mitutoyo calipers. The mice received a single intra-tumor injection of Zika virus (2×10⁶ pfu) on Day 29. Mice were treated with a vehicle injection for comparison as a control group. The vehicle solution may be composed of Sterile Dulbecco's phosphate-buffered saline (DPBS). The DPBS solution contains 2.67 mM of potassium chloride (KCl), 1.47 mM potassium phosphate monobasic (KH₂PO₄), 137.93 mM sodium chloride (NaCl), and 8.06 mM sodium phosphate dibasic (Na₂HPO₄-7H₂O). The composition is at a pH of 7.0-7.3. The tumor sizes and weights were monitored through Day 38 and continuously measured in size every two weeks with the use of Mitutoyo calipers. The tumor size measurements are illustrated in FIG. 7, the tumor weights are illustrated in FIG. 8, and the tumor growth fold change of the tumors are illustrated in FIG. 9. The results from FIG. 7 indicate that that the tumors treated with Zika virus had a significant decrease in tumor size in comparison to the tumors treated with the vehicle solution. As illustrated at Day 38, the tumors treated with Zika virus injection had a median size of approximately 450 mm³ while the tumors treated with the vehicle injection had a median size of approximately 1300 mm³. Further, the results of FIG. 8 indicate that the weights of the tumors in mice that were treated with the vehicle injection were higher than the weights of the tumors in the mice that were treated with Zika virus injection. Further, the results of FIG. 9 indicate that the median tumor growth fold change of the tumors treated with Zika virus injection were significantly lower than the mean tumor growth fold change of the tumors treated with the vehicle injection.

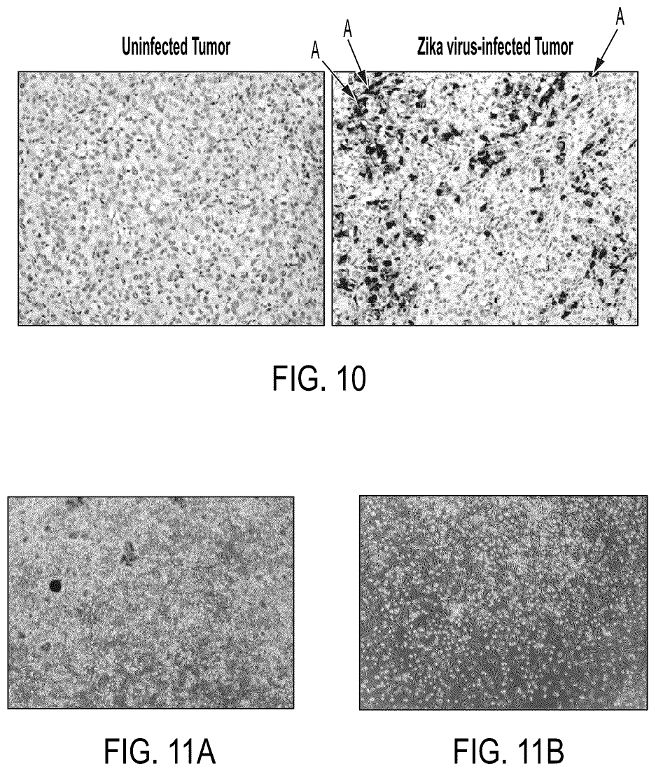

Further, in these experiments, representative immunohistochemistry staining at a magnification of 200× of isolated SkOV3 tumors stained for Zika virus NS2B protein in Zika virus infected tumors and the uninfected tumors was conducted (FIG. 10). The staining is shown in gray scale, with the darkest gray coloration reflecting detection of NS2B, examples of which are shown labeled as “A.” The results indicate that Zika virus-infected tumor expressed the NS2B protein while the uninfected tumor did not. Overall, the results of these experiments suggest that ovarian cancer cells infected with Zika virus are capable of the expressing Zika virus. The results further suggest that ovarian tumor growth is significantly impaired or reduced after Zika virus infection due to lysis induced by Zika virus infection.

Transfection and Infection with Zika Virus

In further experiments, ovarian cancer cells of the SkOV3 cell line were infected with Zika virus at multiplicity of infection (MOI)=0.3 or transfected with purified Zika virus RNA at 0.83 genome copies per cell to explore the effectiveness of transfection of Zika virus. Zika virus RNA was purified from Zika virus MR766, and an isolation and purification yield of 50% was assumed. The purified Zika virus MR766 RNA were transfected into the cultured SkOV3 ovarian cancer cells using Lipofectamine™ MessengerMAX™ (ThermoFisher™/Invitrogen™ https://www.thermofisher.com/order/catalog/product/LMRNA003) as a transfection reagent.

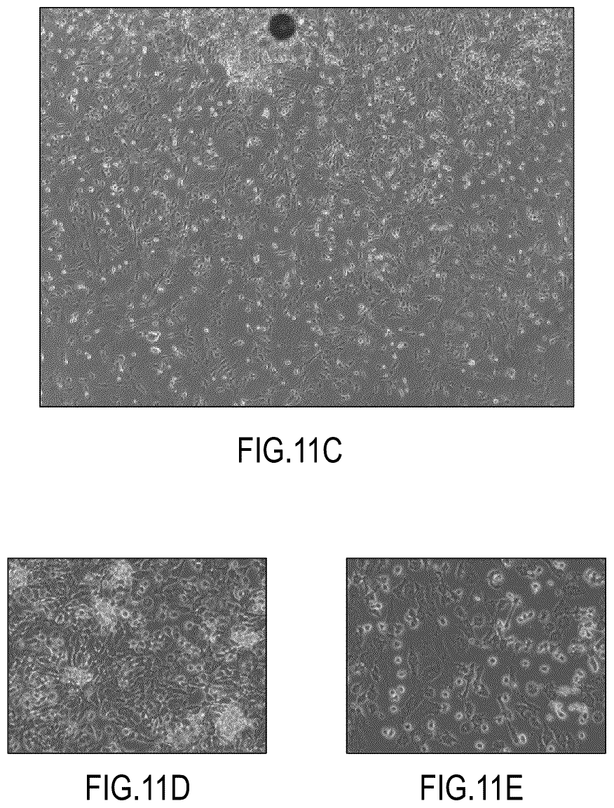

FIG. 11A illustrates a picture taken at 4× magnification of a sample of SkOV3 cells on Day 4 after transfection with 0.1 ug of GFP mRNA as a negative control sample of cells. FIG. 11B illustrates a picture taken at 4× magnification of a sample of SkOV3 cells on Day 4 after infection of the cells with Zika virus at multiplicity of infection (MOI)=0.3. FIG. 11C is a picture taken at 4× magnification of a sample of SkOV3 cells transfected with purified Zika RNA at 0.83 genome copies per cell. FIG. 11D illustrates an image of the sample shown in FIG. 11A at a magnification of 20×. FIG. 11E illustrates an image of the sample shown in FIG. 11B at a magnification of 20×. FIG. 11F illustrates an image of the sample shown in FIG. 11C at a magnification of 20×.

In FIGS. 11B, 11C, 11E, and 11E, the population of the SkOV3 ovarian cancer cells is less than is shown in the negative control samples of FIG. 11A and FIGS. 11D, suggesting that, like Zika virus infection, transfection of purified Zika virus RNA caused a reduction in the ovarian cancer cells of the cells in comparison to the sample of SkOV3 ovarian cancer cells that are not treated with Zika virus or RNA.

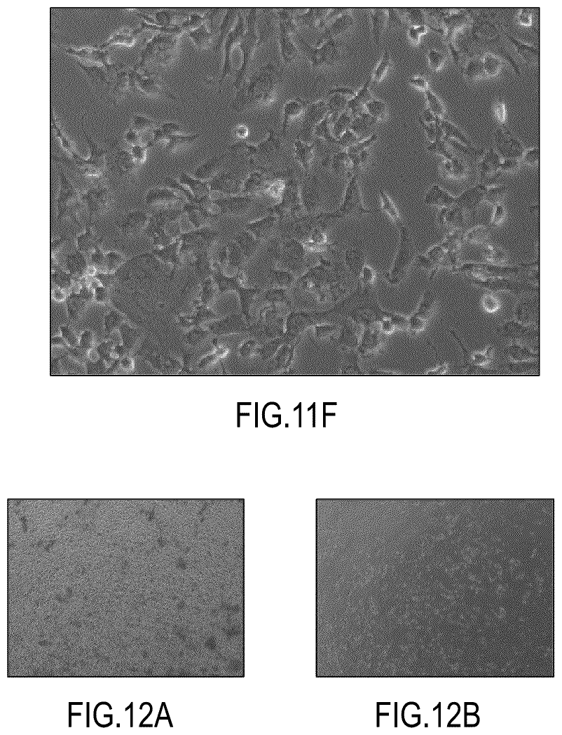

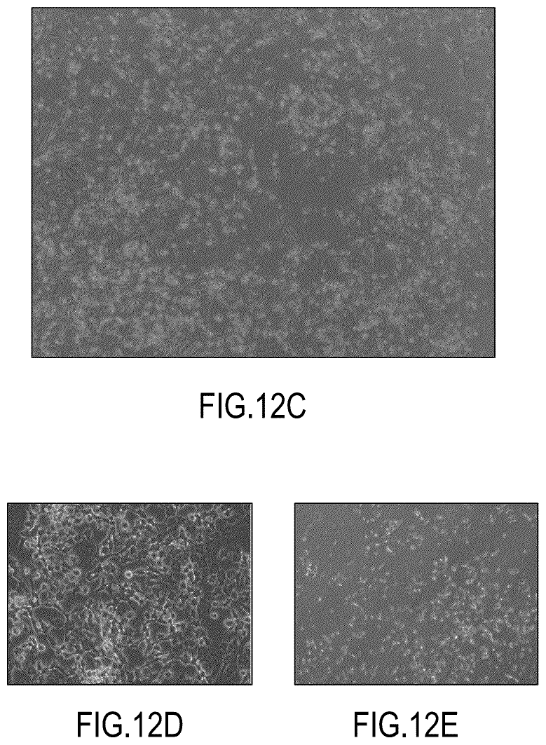

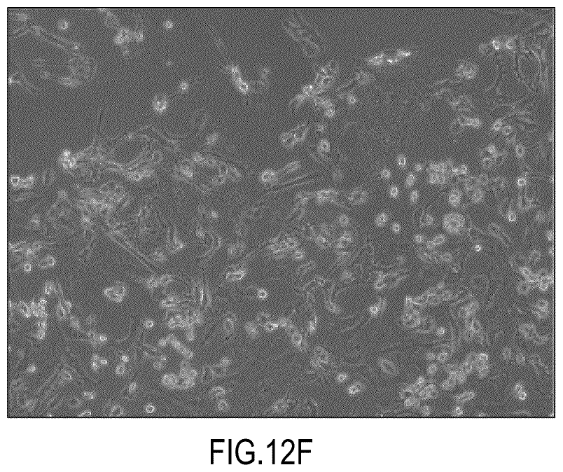

FIG. 12A is an image of the sample of the FIG. 11A taken at 4× magnification and at Day 6 following transfection with 0.1 ug of GFP mRNA. FIG. 12B is an image of the sample shown in FIG. 11B taken at 4× magnification at Day 6 following infection with Zika virus at multiplicity of infection (MOI)=3. Further, FIG. 12C is an image of the sample of FIG. 12B at 4× magnification at Day 6 following transfection with Zika RNA at 0.83 genome copies per cell. Additionally, FIGS. 12D-12F are images of the samples shown in FIGS. 12A-12C, respectively, at 20× magnification. The results of FIGS. 12B, 12C, 12E, and 12F indicate that like Zika virus infection, transfection of purified Zika virus RNA caused a reduction in the ovarian cancer cells of the cells in comparison to the sample of SkOV3 ovarian cancer cells that are not treated with Zika virus or RNA. The population of the ovarian cancer cells is reduced after the treatment with Zika virus in the comparison to the ovarian cancer cells that are not infected with Zika virus, which are shown in FIGS. 12A and 12D.

These experiments indicate that transfection of the entire Zika virus genome into permissive cells would lead to the production of replication-competent virus. Further, overall, these results indicate that that the ovarian cancer cells, whether transfected with purified Zika virus RNA or infected with Zika virus, are reduced in population as compared to when the cells are not transfected nor infected with Zika virus.

### Effect of Nicaragua and Brazil Viruses on Inhibiting Growth of Ovarian Tumors

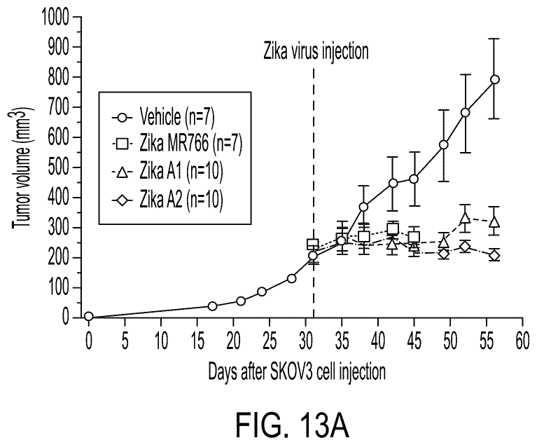

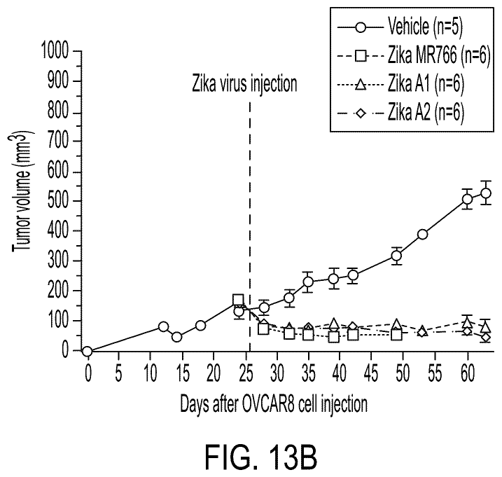

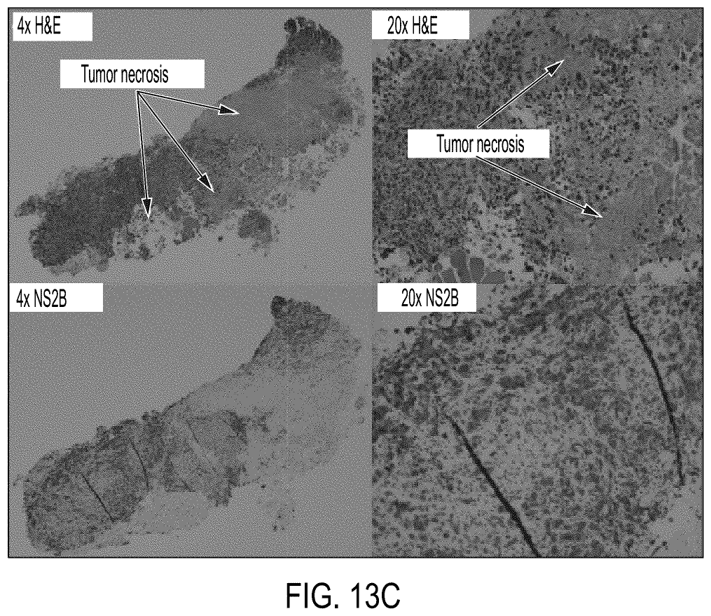

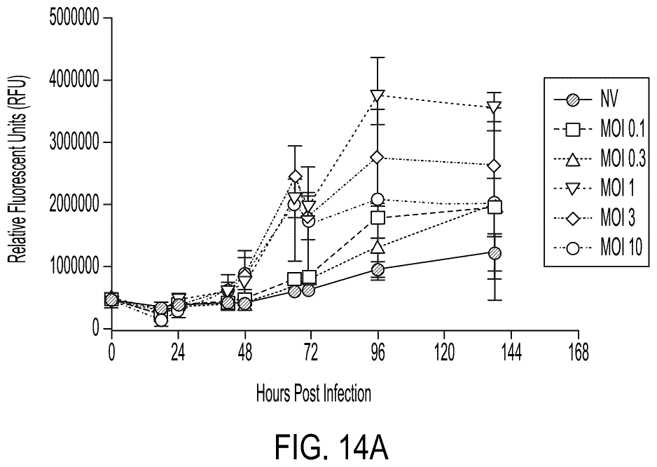

Zika virus strains MR766, Brazil-SJRP/2016-184, and Nicaragua/2016 UCB 7420 were injected into subcutaneous SKOV3 and OVCAR8 tumors created in female NSG mice. Cultured RD cells (a cell line derived from a child with embryonal rhabdomyosarcoma) were highly susceptible to Zika virus-induced lysis; infected cells die within 48 hours of exposure to MOI=0.1 MR766. FIG. 13A and FIG. 13B depict graphs with tumor volumes are plotted as a function of virus treatment. FIG. 13C provides H&E and viral NS2B staining (red) of virus-treated OVCAR8 tumors.

The results indicate that cultured RD cells are highly susceptible to zika virus induced lysis. The cells exhibit morphologic findings characteristic of infected cells. Because the cells are rapidly killed by Zika virus, they will be advanced into animal studies.

### Zika Virus MR766-Induced Lysis on Rhabdomyosarcoma (RD) Cells

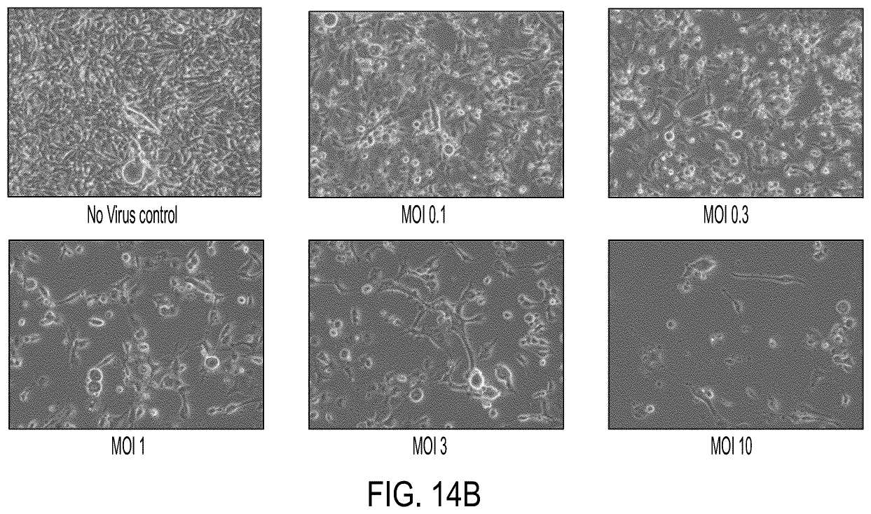

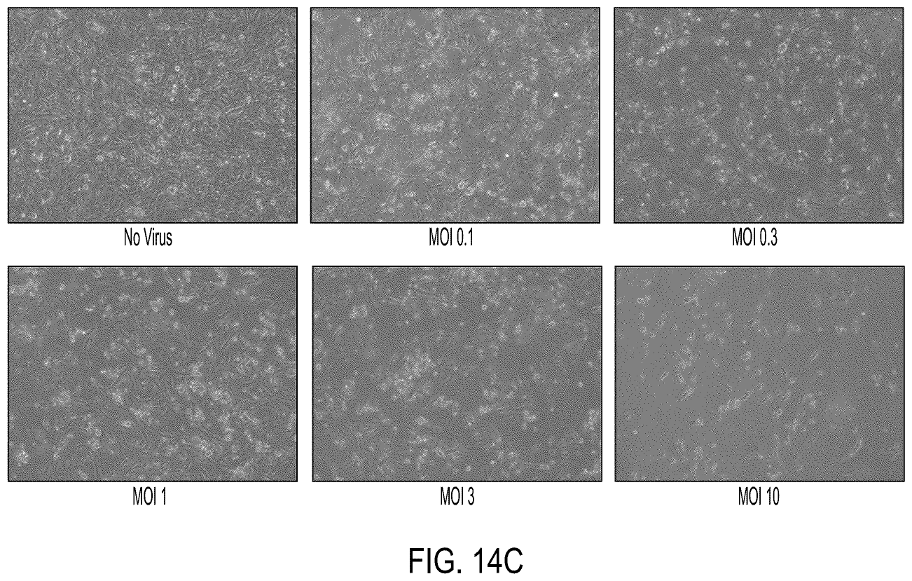

Rhabdomyosarcoma (RD) cells were infected with specified amounts of purified and titered viruses MR766 (0, 0.1, 0.3, 1, 3, and 10 plaque-forming units (PFUs) per cell). Cell death was be measured daily using the CellTox™ Green Cytotoxicity Assay (Promega). Cell death reported as fluorescence units and normalized to fluorescence from matched uninfected cells is shown in FIG. 14A.

The cells were assessed and photographed each day by brightfield microscopy. FIG. 14B shows the cytopathic effects of Zika virus infection of cultured RD cells. The images depict 20× brightfield microscopy of Zika virus MR766-infected RD cells. The images were obtained 3 days after infection. FIG. 14C shows the cctopathic effects of Zika virus infection of cultured RD cells. The images depict 20× brightfield microscopy of Zika virus Nicaragua-infected RD cells. The images were obtained 3 days after infection.

### Envelope Proteins on MR766 Infected Rhabdomyosarcoma (RD) Cells

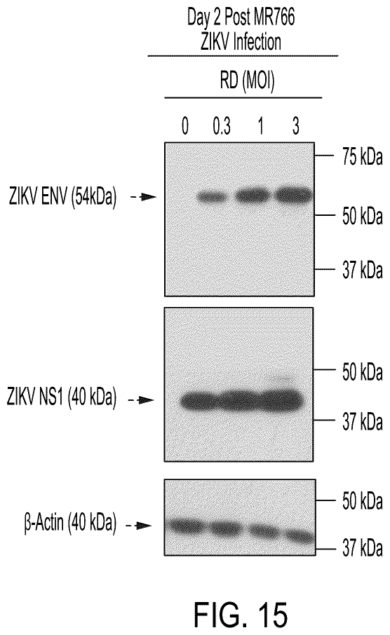

To investigate the earlier stages of viral replication, RD cells were infected with specified multiplicities of infection of Zika virus MR766. After 48 hours, the cells were collected, lysed, and Zika virus NS1 and envelope protein expression were measured by western blot using anti-NS1 and anti-env antibodies. The western blot images are depicted in FIG. 15. Beta-actin was used as a loading control.

The results suggest that Zika virus infected RD cells produce the viral NS-1 and envelope proteins tells us that RD cells support the early stages of viral replication.

### Replication of Zika Virus in RD Cells

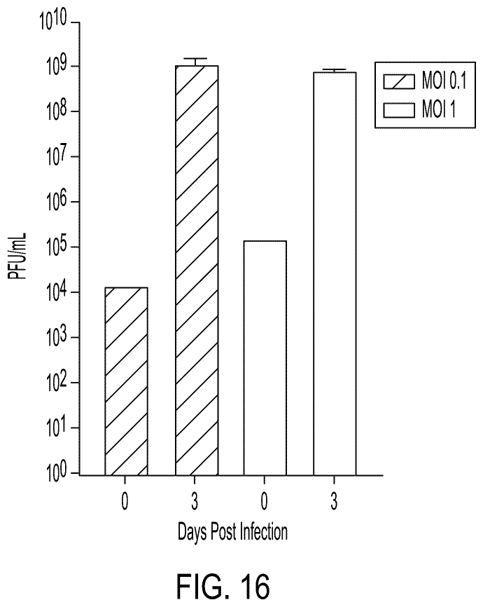

In these experiments, RD cells were infected with the multiplicities of infection of Zika virus MR766 specified in FIG. 16. The results are shown for for 0 or 3 days after infection with a multiplicity of infection (MOI) of 0.1 or 1. Tissue culture supernatants were then assessed by plaque assay to measure the production of replication-competent virus.

The results suggest that RD cells are very effective for replicating fully competent Zika virus. This indicates that murine tumors made with RD cells are likely to be infected easily and, because the tumor cells produce virus, will maintain infection as long as tumor cells are present.

### Expression of AXL and CD24 in RD Cells

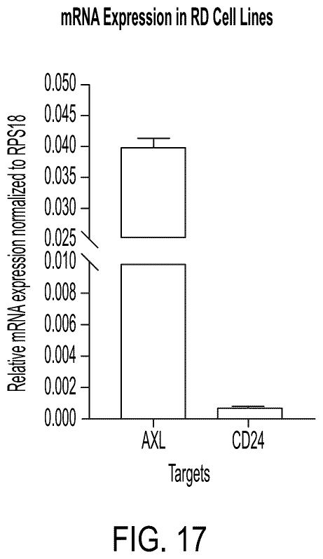

In this experiments real-time PCR (RT-PCR) was performed to compare AXL and CD24 mRNA expression with mRNA expression of the housekeeping gene RPS18. The RNA concentration was standardized. The RPS18 gene was used as the control gene to obtain the relative CD24 and Axl mRNA expression within each cell line. The mRNA expression for each cell line is showed in terms of fold change over RPS18 and then normalized to Axl and CD24 mRNA expression in RD cells (FIG. 17). The levels of relative mRNA expression indicates that the co-expression of AXL and CD24 underlies the ability of Zika virus to infect and lyse RD cells effectively.

### Protocols for Experiments

The experiments for the discussion above were performed as follows.

### Cell Lines Used & Conditions:

The ovarian cancer cells used included SkOV3-Gluc, SkOV3-GFP, SkOV3, ES2, A2780, A2780-cis, and A2780-ADR. SKOV3-GFP-Luc: parental SKOV3 cell line was from ATCC (catalog HTB-77). Dr. Alicja Copik's lab at UCF transduced the parental SKOV3 cell line with the lentiviral construct pLV[Exp]-EGFP:T2A:Puro-EF1A>Luc2 (VectorBuilder, Inc.) stably express EGFP and firefly luciferase. The stable daughter cell line, SKOV3-GFP-Luc, was obtained from Dr. Copik and the DNA short tandem repeat (STR) profile of the daughter cell line was confirmed by the NCH-DE Biobank/Genetics core facility. The STR profile of the daughter cell line matched that of the parental SKOV3 cell line as well as the STR profile reported by ATCC. A2780, A2780-Cis, and A2780-ADR cells were obtained from Sigma Aldrich (catalog numbers 93112519, 93112517, 93112520). ES2 cells were obtained from ATCC (catalog number—CRL1978).

The cell culture conditions of the ovarian cancer cells will be described further. SKOV3-GFP-Luc, A2780, A2780-Cis, A2780-ADR and ES2 cells lines were cultured in Roswell Park Memorial Institute (RPMI) 1640 medium+GlutaMAX (Gibco, ThermoFisher Scientific) supplemented with 10% heat-inactivated fetal bovine serum (FBS; BioTechne) and 1% penicillin-streptomycin.

### Cell Line Infection & Cell Viability Assays:

Cells (SKOV3-GFP-Luc, A2780, A2780-Cis, ES2) were plated in 96-well, clear-bottom black plates and were infected with Zika virus at MOI=1. Along with infected conditions, uninfected controls were used to measure baseline cell death. At the time of infection, the cell lines were treated with CellTox™ Green (1:1000 dilution) and cytotoxicity was measured as fluorescence units across corresponding time points starting from Day 0, for every 24 hours until Day 4. CellTox™ Green Cytotoxicity Assay (Promega) was used to measure the cytotoxicity and fluorescence was read on Spectromax-iD3 plate reader.

### Western Blot Analysis of Zika-Infected Cell Lines:

Cells (SKOV3-GFP-Luc, A2780, A2780-Cis, and ES2 cells) were infected with Zika virus at MOI=1 at around 80% confluency. Two days post-infection, the medium was removed, washed with PBS and the plates were placed on ice. 1 mL of DPBS (supplemented with protease-inhibitor cocktail, PMSF and phosphatase-inhibitor cocktail) was added to the plate and cells were scrapped. The collected cells were lysed with RIPA buffer (50 mM Tris-HCl, pH 8; 150 mM NaCl; 1% IGEPAL; 0.5% sodium deoxycholate; 0.1% SDS). Concentration of total protein was quantified by BCA method and samples were prepared for loading with 5× sample buffer (250 mM tris-HCL, pH 6.8; 50% Glycerol; 10% SDS; 0.5% Bromophenol blue), 2-BME followed by boiling at 95° C. 15 μg of samples were resolved on 8-12% sodium dodecylsulfate-polyacrylamide gel electrophoresis (SDS-PAGE) gels and transferred to nitrocellulose membranes. Samples were then probed with antibodies for Zika-NS1 and Zika-Envelope antibodies (GeneTex). Actin (Cellsignaling) was used as loading control. Blots were then visualized by horseradish-peroxidase-conjugated antibodies and SuperSignal™ West Pico PLUS chemiluminescent substrate (ThermoScientific, Rockford, IL, USA). Densitometry analysis on blots were performed using Image Studio™ (Version 4, LI-COR Biotechnology, Lincoln, NE, USA) as per the manufacturer's instructions.

Quantitative Qualitative PCR of Axl and CD24 mRNA Expression:

Cells (SKOV3-GFP-Luc, A2780, A2780-Cis, ES2) were infected with Zika virus at MOI=1 at around 80% confluency. Two days post-infection, cells were harvested in 250 μL of Tri-reagent. Total RNA was isolated from cells using Direczol RNA miniprep Kit (Zymoresearch) using manufacturer's protocol and RNA concentrations were determined by UV spectrophotometry (Thermofisher). Approximately 1 μg of RNA was converted into cDNA using the Applied Biosystems High Capacity cDNA RT kit. 20 ng of cDNA was then used in qPCR and the expression levels of AXL and CD24 were analyzed. ESD was used as the house-keeping gene. The PCR oligo sequences are as follows: pan-CD24 exon 2 sense primer (5′-TCAAGTAACTCCTCCCAGAGTA-3′), pan-CD24 exon 2 anti-sense primer (5′-AGAGAGTGAGACCACGAAGA-3′), and pan-CD24 probe (/56-FAM/CCCAAATCCAACTAATGCCACCACC/3IABkFQ/); AXL sense primer (5-GTCCTCATCTTGGCTCTCTTC-3′), and AXL anti-sense primer (5′-GACTACCAGTTCACCTCTTTCC-3′); and ESD sense primer (5′-TCAGTCTGCTTCAGAACATGG-3′) and ESD anti-sense primer (5′-CCTTTAATATTGCAGCCACGA-3′).

### Zika Virus Propagation & Purification Methods:

Vero cells that were maintained in Dulbecco's Modified Eagle Medium (DMEM) with 10% FBS and 1% Pen-Strep. When they reached around 80% confluency, the medium was removed, and the cells were washed with DPBS twice. Cells were then infected with Zika virus (MR766) at MOI of 0.001 diluted in infection medium (plain DMEM medium without FBS or Pen-Strep; 5% BSA). The plates were incubated at 37° C. for 2 hours after which 12 mL of replacement medium (DMEM with 2% FBS and 1% Pen-Strep) was added to each plate. Three days post-infection, when cells began to exhibit cytopathic effects of the virus, the supernatant was collected and centrifuged at 3000 rpm for 15 minutes at 15° C. to remove debris. Virus in the supernatant was concentrated by doing ultracentrifugation at 24000 RPM at 15° C. for 3 hours. The pellet is resuspended in DMEM (without FBS or Pen-Strep). The virus is stored at −80° C. after snap-freezing them with liquid nitrogen. Viral titer is determined by performing plaque assay.

### Plaque Assay:

ZIKV viral titers were determined on Vero cells using plaque assays. 6-well plates of Vero cells were infected with serial dilutions of virus in DMEM supplemented with 2% HI FBS. After 3 hours of incubation, cells were washed with PBS and overlayed with a 1:1 solution mixture of 0.6% agarose and DMEM supplemented with 2% HI FBS and 1% Pen-Strep. After the overlay had solidified, plates were incubated at 37° C. for 4 days. The plates were then stained with crystal violet to observe the plaques.

### Purified Zika RNA Protocol:

RNA was extracted from purified viral particles using the Trizol-Chloroform protocol. Purified Zika virus was lysed using Trizol. Then, an appropriate volume of chloroform was added to separate the three phases (Clear RNA phase, white DNA phase and organic protein phase). Clear RNA layer was transferred to a new tube and RNA was precipitated with isopropanol. Glycogen was used as a carrier. RNA was quantified using UV spectroscopy (ThermoFisher).

### Zika RNA Genome Liposomal Preparation and Transfection Protocol:

Lipofectamine MessengerMax (ThermoFisher) was used to transfect the viral RNA in SKOV3 cells. Liposomes were prepared as per the manufacturer's protocol with purified viral RNA and 0.1 g of EGFP mRNA (Cleancap EGFP mRNA, Trilink Biotechnologies) was used as a positive control for transfection. Cells were monitored and pictures were taken with EVOS M5000 microscope (ThermoFisher) to observe and record the cytopathic effects. The cells were monitored in a humidified 5% CO2 incubator for six days (d). The agarose overlap was removed, and cells were fixed and stained for 30 min (minute) with a 1% crystal violet solution containing 3.7% formaldehyde, 20% ethanol, and PBS.

The disclosures of each and every patent, patent application, GenBank record, and publication cited herein are hereby incorporated herein by reference in their entirety.

While the disclosure has been disclosed with reference to specific embodiments, it is apparent that other embodiments and variations of this disclosure may be devised by others skilled in the art without departing from the true spirit and scope used in the practice of the disclosure. The appended claims are intended to be construed to include all such embodiments and equivalent variations.

1: A pharmaceutical composition for treatment of a tumor expressing at least one of a plurality of receptor proteins, the plurality of receptor proteins including CD24, Axl, DC-SIGN, Tyro3, MER, MerTK TIM-1, TIM-4, TLR3, TLR8, RIG-1, and MDA5, the pharmaceutical composition comprising:
(a) a purified viral RNA of an oncolytic Zika virus in a liposome and a pharmaceutically acceptable carrier, or
(b) a pharmaceutically acceptable carrier and a unit dosage form of an oncolytic Zika virus.

2: The pharmaceutical composition of claim 1, wherein the purified RNA of the oncolytic Zika virus or the oncolytic Zika virus is in an amount sufficient to reduce the size of the tumor upon administration to a subject in need of treatment for the tumor.

3: The pharmaceutical composition of claim 1, wherein the pharmaceutical composition is formulated for intertumoral administration, intradermal administration, intracranial administration, intraventricular administration, intraocular administration, intrathecal administration, subcutaneous administration, or intravenous administration.

4: A combination therapy comprising the pharmaceutical composition of claim 1 and an anti-neoplasm treatment selected from the group consisting of: a chemotherapy, a radiotherapy, surgery, a hormone therapy, an immunotherapy, and a combination thereof.

5: A method for treating a disease characterized by expression of a putative Zika virus receptor protein in an individual in need of such treatment comprising:
administering the pharmaceutical composition of claim 1, wherein the disease is characterized by expression of a putative Zika virus receptor protein.

6: The method of claim 5, wherein the disease is a tumor selected from the group of: an ovarian cancer, a rhabdomyosarcoma, a colorectal cancer, a B cell lymphoma, erythroleukemia, a glioma, a non-small cell lung cancer, an esophageal squamous cell carcinoma, a hepatocellular carcinoma, a hepatoblastoma, a cholangiocarcinoma, a pancreatic adenocarcinoma, a melanoma, an urothelial carcinoma, a breast cancer, a primary neuroendocrine carcinoma, a neural sheath tumor, a peripheral nervous system tumor, a neurofibroma, a schwannoma, a neural crest-derived tumor, an HPV-associated malignancy, an Epstein-Barr virus-induced malignancy, and a prostate carcinoma.

7: The method of claim 6, wherein the tumor expresses at least one of CD24, Axl, DC-SIGN, Tyro3, MER, MerTK, TIM-1, TIM-4, TLR3, TLR8, RIG-1, and MDA5.

8: The method of claim 7, wherein the tumor is a resistant or a refractory tumor.

9: The method of claim 8, wherein the tumor is a resistant or a refractory tumor due to continued treatment of the tumor with anti-neoplasm treatment.

10: A pharmaceutical composition for treatment of a tumor expressing at least one putative Zika virus receptor protein, the pharmaceutical composition comprising:
(a) a purified viral RNA of an oncolytic Zika virus in a liposome and a pharmaceutically acceptable carrier, or
(b) a pharmaceutically acceptable carrier and a unit dosage form of an oncolytic Zika virus.

11: The pharmaceutical composition of claim 10, wherein the at least one putative Zika virus receptor protein is one of CD24, Axl, DC-SIGN, Tyro3, MER, MerTK, TIM-1, TIM-4, TLR3, TLR8, RIG-1, and MDA5.

12: The pharmaceutical composition of claim 10, wherein the purified RNA of the oncolytic Zika virus or the oncolytic Zika virus is in an amount sufficient to reduce the size of the tumor upon administration to a subject in need of treatment for the tumor.

13: The pharmaceutical composition of claim 10, wherein the pharmaceutical composition is formulated for intertumoral administration, intradermal administration, intracranial administration, intraventricular administration, intraocular administration, intrathecal administration, subcutaneous administration, or intravenous administration.

14: A combination therapy comprising the pharmaceutical composition of claim 10 and an anti-neoplasm treatment selected from the group consisting of: a chemotherapy, a radiotherapy, surgery, a hormone therapy, an immunotherapy, and a combination thereof.

15: A method for treating a disease characterized by expression of a putative Zika virus receptor protein in an individual in need of such treatment comprising:
administering the pharmaceutical composition of claim 10, wherein the disease is characterized by expression of a putative Zika virus receptor protein.

16: The method of claim 15, wherein the disease is a tumor selected from the group of: an ovarian cancer, a rhabdomyosarcoma, a colorectal cancer, a B cell lymphoma, erythroleukemia, a glioma, a non-small cell lung cancer, an esophageal squamous cell carcinoma, a hepatocellular carcinoma, a hepatoblastoma, a cholangiocarcinoma, a pancreatic adenocarcinoma, a melanoma, an urothelial carcinoma, a breast cancer, a primary neuroendocrine carcinoma, a neural sheath tumor, a peripheral nervous system tumor, a neurofibroma, a schwannoma, a neural crest-derived tumor, an HPV-associated malignancy, an Epstein-Barr virus-induced malignancy, and a prostate carcinoma.
